(19) United States
(12) Patent Application Publication
(10) Pub. No.: US 2023/0355805 A1
(43) Pub. Date: Nov. 9, 2023

(54) ENGINEERED EXTRACELLULAR VESICLES DISPLAYING ENHANCED PHARMACOKINETICS

(71) Applicant: Evox Therapeutics Ltd., Oxford Oxfordshire (GB)

(72) Inventors: Dhanu GUPTA, Huddinge (SE); Samir EL ANDALOUSSI, Huddinge (SE); Oscar WIKLANDER, Huddinge (SE); Joel NORDIN, Huddinge (SE)

(21) Appl. No.: 18/028,576

(22) PCT Filed: Sep. 29, 2021
(86) PCT No.: PCT/EP2021/076845

§ 371 Date: Mar. 27, 2023

(30) Foreign Application Priority Data
Sep. 29, 2020 (GB) 2015399.5

(51) Int. Cl. A61K 48/00; A61K 47/69; A61K 9/50; C07K 14/47; C07K 14/54; C07K 14/705; C07K 14/715; C12N 15/88

(52) U.S. Cl. CPC A61K 48/0041; A61K 47/6901; A61K 48/0033; A61K 9/5068; C07K 14/4702; C07K 14/5412; C07K 14/70582; C07K 14/70596; C07K 14/7151; C12N 15/88

(57) ABSTRACT

The present invention relates to engineered extracellular vesicles (EVs) as a therapeutic modality for the treatment of various severe diseases. More specifically, the invention relates to a novel approach to manufacturing engineered EVs which extends their half-life and alters their biodistribution, resulting in a novel therapeutic modality able to carry various types of drugs suitable for application in not only genetic diseases but more broadly across essentially all therapeutic areas.

### FIELD OF THE INVENTION

The present invention relates to engineered extracellular vesicles (EVs) as a therapeutic modality for the treatment of various severe diseases. More specifically, the invention relates to in vivo production of EVs in order to modulate the pharmacokinetics and biodistribution of EVs.

### BACKGROUND ART

Extracellular vesicles (such as exosomes) are nanometer-sized vesicles produced by most cell types and functioning as the body's natural transport system for proteins and peptides, nucleic acids, lipids, and various other biomolecules between cells. EVs have a number of potential therapeutic uses and engineered EVs are already being investigated as delivery vehicles for protein biologics, nucleic acid therapeutics, gene editing agents and small molecule drugs. However, as EVs are rapidly taken up into target tissues clinical application of exosome therapeutics will likely require relatively frequent dosing. EVs (that is, a population of a given EV, whether engineered or not) normally have a plasma half-life in the range of minutes in mammals and this short half-life is especially evident upon systemic intravenous administration.

EV manufacturing have come a long way since the initial academic publications that pioneered the field of exosome therapeutics (for instance, Alvarez-Erviti et al., Nature Biotechnology, 2011). However, scaling up of manufacturing and upstream and downstream processing (including purification) of EVs still require further development to inter alia reduce the changes imparted on the EV surface during purification and be ready for commercial use in large patient populations.

Despite these disadvantages EVs still hold significant potential as a novel drug modality for delivery of drug cargo molecules across biological barriers and the modularity of EV therapeutics is an unrivalled advantage of EVs over other modalities and delivery systems. The present invention addresses the issues of short plasma half-life and the challenges related to EV manufacturing at scale, while maintaining the delivery properties of EVs and their inherent modularity, thereby enabling a new approach to EV based therapeutics.

### SUMMARY OF THE INVENTION

It is hence an object of the present invention to overcome the above-identified problems associated with half-life, biodistribution and manufacturing of EVs.

In a first aspect, the present invention relates to a composition comprising a delivery vector which comprises a polynucleotide cargo coding for a fusion protein, wherein translation of the polynucleotide cargo into the corresponding fusion protein results in the production of at least one EV comprising said fusion protein. The translation of the polynucleotide cargo by a cell in vivo results in the production of genetically engineered EVs comprising the fusion protein in question, resulting in the in situ production of a genetically engineered delivery modality in the form of an engineered, autologous EV carrying the fusion protein. Importantly, the fusion protein comprises a protein of interest (POI) that is either itself a drug (for example but not limited to an enzyme, a transporter, a transcription factor, a chaperone, etc.) or that has the ability to bind to a drug (for example but not limited to an mRNA, an shRNA, etc.) and transport it into the engineered EV for subsequent delivery.

In a second aspect, the instant invention relates to a pharmaceutical composition comprising the compositions as described herein (i.e. the composition comprising a delivery vector and the polynucleotide encoding for the fusion protein which when the polynucleotide is expressed leads to translation of the fusion protein and generation of engineered (i.e. modified) EVs comprising the fusion protein).

In a third aspect, the present invention relates to the composition as per the present invention for use in medicine. More specifically, the compositions herein may be for use in the treatment of essentially any disease, disorder, condition, or ailment, preferably selected from the group consisting of genetic diseases, hereditary diseases (including both genetic diseases and non-genetic hereditary diseases), lysosomal storage disorders, inborn errors of metabolism, urea cycle disorders, neuromuscular diseases, neurodegenerative diseases, cancer, autoimmune diseases, cardiovascular diseases, central nervous system diseases, infectious diseases, and inflammatory diseases. In a further aspect, the present invention relates to a method of manufacturing the compositions herein.

In yet another aspect, the present invention relates to a method of producing at least one genetically engineered EV comprising a fusion protein comprising an EV polypeptide and a POI in a mammalian cell. This method comprises contacting the mammalian cell (for instance a human cell) with a composition as described herein, wherein the mammalian cell is capable of translating the polynucleotide cargo into the corresponding fusion protein resulting in the production of mammalian cell-derived EVs comprising the fusion protein and thereby the POI. As abovementioned, the mammalian cell may be any cell of the body of a mammal, for instance a liver cell such as a hepatocyte or a liver macrophage (e.g. a Kupffer cell). Various other cells and cell types in other organs than the liver may also function as “in situ bioreactors” for the essentially autologous yet genetically modified EVs of the present invention.

In a further aspect, the present invention relates to a method of producing patient-derived EVs comprising a fusion protein, with the fusion protein comprising at least one EV polypeptide and at least one POI, the method comprising the step of administering to the cells of a patient a composition as per the present invention, whereby the cells of the patient produce the patient-derived EVs. The patient-derived EVs are thus produced in-vivo and are genetically modified patient-derived EVs. These patient-derived EVs are heterologous to the patient, as a result of the fact that they result from the expression of the designed polynucleotide into the translated fusion protein which in turn comprises the POI (which too may be heterologous to the patient). However, importantly, despite the fusion protein and/or the POI (and/or any other drug bound by the POI) being heterologous to the patient, the EVs are at the same time autologous in the sense that they are produced by the patient for the patient. This has numerous advantages, including high yield as the “normal” cellular machinery is utilised for the expression/production of the engineered EVs, immune-silence due to the autologous EV profile, which is surmised to lead to broad biodistribution, long half-life in the circulation, as well as efficient barrier crossing and drug delivery. In-vivo production of genetically engineered EVs is beneficial as compared to the delivery of a cargo by administration to a patient of an EV produced ex-vivo because a) the EVs are not damaged by the purification process and thus retain their full corona of native proteins and are therefore more likely to be highly biologically active as this will benefit the uptake of the EVs by recipient cells and b) the in-vitro purification process of EVs inevitably excludes certain populations of EVs which are either too large or too small. When EVs are produced in-vivo by this in-situ method the full spectrum of EVs is produced and released by the patient cells, this ensures that a wide range of cargos can be loaded because it is known that certain fusion proteins are preferentially incorporated into certain sized EVs. Thus this in-situ delivery method enables a versatile platform technology for delivery of a very wide range of cargos without the need to alter the purification process for each product. Thus, in a further aspect, the present invention relates to a patient-derived EV (and by default also a population of such EVs) comprising a fusion protein comprising at least one EV polypeptide and at least one POI, wherein the patient-derived EV is manufactured by the method as described above. The present invention further relates to such genetically engineered, patient-derived EVs for use in medicine, in numerous diseases as described herein.

In another aspect, the present invention relates to a method of treatment of a disease, disorder or condition in a subject in need thereof, wherein said method comprises administering to a subject the compositions herein, wherein translation of the polynucleotide cargo into the corresponding fusion protein results in the production of at least one EV comprising the fusion protein comprising a POI. Any disease, disorder or condition is contemplated as a suitable target for the treatment.

In a further aspect, the present invention relates to a method of treating a genetic disease, disorder or condition resulting from a defect gene. Gene defects can take many forms, including mutations, deletions, truncations, duplications, chromosomal damage, deletion or duplication, and gene defects may be monogenic or polygenic. Monogenic genetic defects are particularly suitable for treatment with the patient-derived genetically engineered POI-carrying EVs of the present invention. The method for treating a disease resulting from a gene defect comprises administering to a subject a composition as per the present invention, wherein expression/translation of the polynucleotide cargo into the corresponding fusion protein results in the production of at least one extracellular vesicle (EV) comprising a POI, wherein the POI is a protein corresponding to the defective gene of the subject.

In another aspect, the present invention relates to a method of delivering a POI to a target cell, a target organ or organ system, a target compartment, or a target tissue of a patient. The method of delivering a POI comprises the step of administering to cells (often referred to as producer cells) of a patient the compositions according to the present invention, whereby the producer cells of the patient produces patient-derived EVs comprising a fusion protein comprising the POI, wherein the patient-derived EVs deliver the POI to the target cell. Highly surprisingly, the inventors have discovered that the genetically engineered patient-derived EVs per the present invention have a considerably longer half-life in the circulation as compared to ex vivo-produced genetically engineered EVs (even as compared to ex vivo-produced patient-derived genetically engineered EVs). This surprising technical effect is likely a function of the fact that the EVs are patient-specific (autologous) in combination with them being produced in vivo (also called in situ) in the body of the patient, which is surmised to result in a patient-specific corona associating with the genetically engineered EVs as soon as they enter the systemic circulation, for instance via the blood. The formation of a protein corona in the host (i.e. an autologous corona) is surmised, without wishing to be bound by any theory, to lead to the engineered autologous EV (with a heterologous cargo molecule) being immuno-silent, resulting in a remarkably long plasma half-life in the patient. As an example, the half-life of a population of the genetically engineered subject-derived EVs is normally more than 24 hours, which is at least 10 times as long as the half-life of the corresponding in vitro-manufactured EVs, more preferably 100 times as long. However, the present inventors have observed plasma half-life in vivo to extend beyond 72 hours and even longer. Once again the benefits of in-vivo production of EVs vs purified EVs applies and explains the greater therapeutic effect observed by in-situ produced EVs as compared to ex-vivo produced EVs. In brief these benefits include a) the EVs are not damaged by the purification process and thus retain their full corona of native proteins and are therefore more likely to be highly biologically active and b) there is no loss of any EV sub-populations meaning that effective delivery of a range of different cargos (POIs) is possible due to the versatility of the platform technology.

The present invention as briefly summarised here and as described in more detail below is based on a remarkable feat of cellular engineering resulting in production in situ of genetically engineered, subject-derived (i.e. autologous) EVs carrying a fusion protein comprising a drug in the form of e.g. a POI. This invention represents a completely novel approach to engineered EV therapeutics and allows for less frequent dosing, lower cost of goods, enhanced PK/PD profile and biodistribution, and also enables scalable manufacturing and application of autologous engineered EVs, which is a step-change in terms of engineered EV therapeutics development.

### BRIEF DESCRIPTION OF THE FIGURES

FIG. 1: Schematic diagram explaining the in situ engineered EV production concept, whereby transiently engineered patient cells in vivo produce engineered EVs, i.e. harnessing the patient's own ability to produce exosomes to deliver drugs to hard-to-reach organs which results in long-lasting sustained engineered fusion protein-carrying EV production of otherwise patient-specific, autologous EVs engineered to contain a desired drug (in the form of a protein of interest (POI)) and optionally additional moieties to enhance the pharmacological activity of the engineered EV.

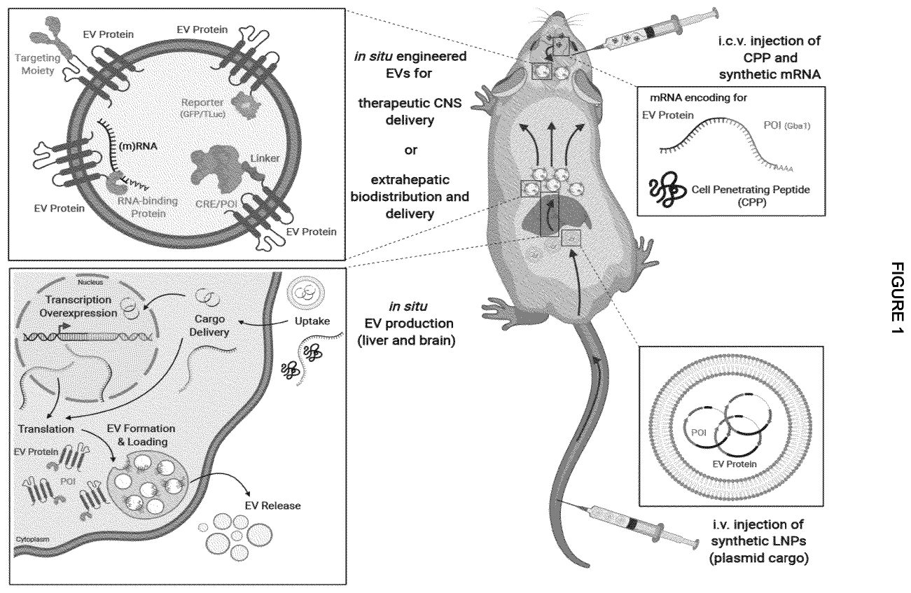

FIG. 2: In vivo data showing that therapeutic genetically engineered EVs produced in situ are capable of providing long term therapeutic effect in a mouse model of colitis.

FIG. 3: In vivo biodistribution data showing that EVs produced in situ in the liver are detectable in wide range of organs and for extended time periods in plasma.

FIG. 4: Comparison of the level of enzymatic activity in mouse plasma over time of (i) mouse in situ produced EVs transiently genetically engineered to be secreted from the mouse cells and to carry a fusion protein comprising human CD63 and the enzyme NanoLuciferase as the POI, and (ii) administration of in vitro-manufactured EVs carrying the exact same fusion protein, demonstrating that autologous subject-specific EVs carrying the fusion protein comprising the POI have significantly improved half-life as compared to ex vivo produced EVs.

FIG. 5: In vivo biodistribution and half-life data showing that the addition of an albumin binding domain into the fusion protein which comprises the EV polypeptide and the POI extends the half-life of in situ-produced genetically engineered EVs produced in situ even further.

FIG. 6: In-vivo biodistribution of EVs expressing nanoluc fusion proteins (human CD63-luc, human CD63-ABD-luc or luc alone) following in-situ exosome production via delivery of mRNA by lipid nanoparticle (LNP).

FIG. 7: Effect of albumin-binding polypeptides on half-life of in situ engineered EVs produced following mRNA delivery by LNP.

FIG. 8: Comparison of plasma kinetics of in-situ vs purified EVs.

FIG. 9: Evidence that fusion proteins comprising a range of different EV polypeptides are also capable of delivering cargos.

FIG. 10: In-situ EV delivery of therapeutic super-repressor ikBa protein for treatment of colitis.

FIG. 11: Evidence that in-situ produced EVs reduce inflammatory cytokine levels in colitis model.

### DETAILED DESCRIPTION OF THE INVENTION

The present invention relates to a novel and inventive approach to the development of EV therapeutics, which addresses the principal drawbacks of EV based drug development. Instead of manufacturing engineered EV therapeutics ex vivo in in vitro cell culture the inventors behind the present invention have made the remarkable invention that by genetically engineering patient-derived EVs in situ in the in vivo setting, these genetically engineered EVs behave differently to ex vivo-manufactured EVs as it relates to both pharmacokinetics and biodistribution. The rapid plasma clearance seen when administering in vitro manufactured EVs from primary cells or cell lines is completed abrogated when, as the present inventors have shown, genetically engineered EVs carrying a given drug cargo are created in situ by turning a target cell existing in an organ system in vivo into a bioreactor for EV production. This is achieved by the inventive method of delivering a polynucleotide construct encoding for a fusion protein comprising at least an EV polypeptide and a protein of interest (POI) into a target cell. Upon delivery of the polynucleotide construct, the target cell machinery translates (and transcribes when so required, i.e. when the polynucleotide construct is not an mRNA) the polynucleotide construct into the fusion protein, resulting in the production and ultimately secretion from the cell of genetically engineered EVs comprising said fusion protein. The POI which is comprised in the protein may itself be a drug (e.g. a therapeutic enzyme for enzyme replacement therapy) or may bind to a drug, i.e. a pharmacologically active agent, such as another protein or a nucleic acid, etc. This results in the production (either transiently and stably, depending on the polynucleotide and the delivery vector) of POI-carrying genetically engineered EVs which are secreted by the patient from the target cells into the circulation, where these EVs have a considerably longer circulating half-life and a different biodistribution profile than ex vivo produced genetically engineered EVs.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the fusion proteins described herein in connection with the compositions comprising polynucleotides encoding such fusion proteins are to be understood to be disclosed, relevant, applicable and compatible with all other aspects, teachings and embodiments herein, for instance aspects and/or embodiments relating to the genetically engineered EVs and/or their application in medicine. Furthermore, certain embodiments described in connection with certain aspects, for instance the different viral and non-viral delivery vectors as described in relation to aspects pertaining to the compositions and pharmaceutical compositions are to be understood to be disclosed, relevant, applicable and compatible with all other aspects and/or embodiments, such as those pertaining to methods of treatment and/or medical uses of such compositions. Furthermore, all EV polypeptides and proteins of interest (POIs) mentioned herein can be freely combined in fusion proteins in any order, sequence, or using any domains, regions, or stretches thereof, using conventional strategies for creation fusion proteins. As a non-limiting example, the EV polypeptides described herein may be freely combined in any combination with one or more POI, optionally combined with other polypeptide domains, regions, sequences, peptides, and groups herein, e.g. linker sequences, self-cleaving domains, endosomal escape domains, RNA-binding domains, targeting moieties, and/or domains which mediate binding to plasma proteins, etc. Moreover, any and all features (for instance any and all members of a Markush group) can be freely combined with any and all other features (for instance any and all members of any other Markush group), e.g. any EV polypeptide may be combined with any POI, which in turn may be combined, used, or applied in combination with any other polypeptide domain, other drug cargo such as a nucleic acid drug cargo (e.g. an RNA molecule such as an mRNA, shRNA, miRNA, self-amplifying RNA etc.) or any other aspects and/or embodiment herein. Furthermore, when teachings herein refer to EVs in singular and/or to EVs as discrete nanoparticle-like vesicles it should be understood that all such teachings are equally relevant for and applicable to a plurality of EVs and populations of EVs. As a general remark, the EV polypeptides, the POIs, additional polypeptides domains and moieties (for instance but not limited to targeting domains, cleavable domains, RNA-binding domains, self-cleaving domains, endosomal escape domains, plasma protein-binding domains, linkers, etc.) and all other aspects, embodiments, and alternatives in accordance with the present invention may be freely combined in any and all possible combinations without deviating from the scope and the gist of the present invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the desired technical effect associated therewith. As long as their biological properties are maintained the polypeptide and/or polynucleotide sequences according to the present application may deviate with as much as 50% (calculated using, for instance, BLAST or ClustalW) as compared to the native sequence, although a sequence identity or similarity that is as high as possible is preferable (for instance 60%, 70%, 80%, or e.g. 90% or higher). Standard methods in the art may be used to determine homology. For example, PILEUP and BLAST algorithms can be used to calculate homology or align sequences to determine identity or similarity. The combination (i.e. fusion) of several polypeptides implies that certain segments of the respective polypeptides may be replaced, truncated and/or modified and/or that the sequences may be interrupted by insertion of other amino acid stretches, meaning that the deviation from the native sequence may be considerable as long as the key properties (e.g. in the context of an EV polypeptide its ability to transport a fusion protein to an EV, or, in the context of a POI that is an enzyme its enzymatic activity) are conserved or at least substantially maintained. Similar reasoning thus naturally applies to the polynucleotide sequences encoding for such polypeptides, whether those polynucleotide sequences are DNA or RNA or a combination of the two and whether they require transcription and translation or merely translation into the corresponding fusion protein. Any accession numbers or SEQ ID NOs mentioned herein in connection with genes, peptides, polypeptides and proteins shall only be seen as examples and for information only, and all genes, nucleotides, polynucleotides, peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein but also variants and derivatives thereof. All accession numbers referred to herein are UniProtKB accession numbers, and all genes, proteins, polypeptides, peptides, nucleotides and polynucleotides mentioned herein are to be construed according to their conventional meaning as understood by a skilled person.

The terms “EV” or “extracellular vesicle” or “exosome” are used interchangeably herein and shall be understood to relate to any type of vesicle that is obtainable from a cell in any form, for instance a microvesicle, (e.g. any vesicle produced from the plasma membrane of a cell), an exosome (e.g. any vesicle derived from the endosomal, lysosomal and/or endo-lysosomal pathway and/or from the plasma membrane or any other membrane of a cell), ARMMs (arrestin domain containing protein 1 (ARRDC1)-mediated microvesicles, which are a form of microvesicles), etc. Exosomes, microvesicles and ARRDC1-mediated microvesicles (ARMMs) represent particularly preferable EVs, but other EVs may also be advantageous in various circumstances. The EVs, exosomes etc. of the present invention may be genetically modified; the terms “genetically engineered” or simply “modified” or “engineered” may also be used. The terms “genetically modified” and “genetically engineered” EV indicates that the EV is derived from a genetically modified/engineered cell. The genetic engineering of the cell and the resultant genetically engineered EV is typically a consequence of the translation (and if required preceded by transcription) of a polynucleotide which encodes for a fusion protein comprising an EV polypeptide, which when introduced into a cell results in production (by the genetically engineered/modified cell) of a genetically engineered EV comprising said fusion protein.

The sizes of EVs may vary considerably but an EV typically has a nano-sized hydrodynamic radius, i.e. a radius below 1000 nm. Exosomes often have a sized of between 30 and 300 nm, typically in the range between 40 and 250 nm, which is a highly suitable size range for therapeutic purposes. Clearly, EVs may be derived from any cell type in vivo albeit that organs such as the liver are highly productive organs for EV production.

It will be clear to the skilled artisan that when describing medical and scientific uses and applications of the EVs, the present invention normally relates to a plurality of EVs, i.e. a population of EVs which may comprise thousands, millions, billions or even trillions of EVs. EVs may be present in concentrations such as 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml or per litre), or any other number larger, smaller or anywhere in between. In the same vein, the term “population”, which may e.g. relate to a genetically engineered EV comprising a certain fusion protein with a certain POI shall be understood to encompass a plurality of entities which together constitute such a population. In other words, individual EVs when present in a plurality constitute an EV population. Thus, naturally, the present invention pertains both to individual EVs and populations comprising EVs, as will be clear to the skilled person.

The term “polynucleotide” and “polynucleotide cargo” as used interchangeably herein shall be understood to relate to a biopolymer comprising at least 10 nucleotide monomers, which may be in the form of ribonucleic acid (RNA) nucleotides, deoxyribonucleic acid (DNA) nucleotides, any combination of DNA nucleotides and RNA nucleotides, and any modified form of RNA nucleotides and/or DNA nucleotides. Polynucleotides may be single-stranded or double-stranded, and they may be linear or circular, with various secondary and tertiary structures. Any polynucleotide whether naturally occurring or non-naturally occurring shall be understood to be a polynucleotide in the spirit of the present invention. Preferred embodiments of polynucleotides include linear mRNA, circular mRNA, linear DNA, circular DNA, plasmid DNA, linear RNA, circular RNA, doggybone DNA (dbDNA), self-amplifying RNA or DNA, viral genomes (single stranded or double stranded, comprised of RNA or of DNA), or modified versions of any of the above, as well as any other suitable polynucleotide cargo. More specifically, as used herein, an “mRNA” refers to a messenger ribonucleic acid that may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. The mRNAs of the present invention normally have a nucleotide sequence encoding for a polypeptide, which in the context of the present invention is typically a fusion protein, said fusion protein in turn comprising a protein of interest (POI). Translation of an mRNA, for example, in vivo translation of an mRNA inside a mammalian cell, may produce a polypeptide, that is as per the aspects and embodiments of the present invention a fusion protein comprising a POI. In one advantageous embodiment, more than one polynucleotide is comprised in the composition, in order to code for more than one POI (for instance two, three or four or more POIs) or one POI and a second protein or RNA molecule wherein the second protein or RNA molecule is the drug (i.e. has the actual pharmacological/therapeutic activity). As an example, two mRNA may be comprised in the compositions here, or two pDNA plasmids, or one pDNA plasmid and one mRNA polynucleotide, etc. It may in some embodiments be advantageous to have an mRNA code for a fusion protein comprising the POI and a pDNA polynucleotide code for an mRNA molecule with which the POI is designed to interact, in order to drive production of genetically engineered in situ produced patient-specific EVs which comprise the fusion protein including the POI and an mRNA which is transported into the engineered EV by virtue of the POI. As can be seen from the above, numerous aspects, alternatives and variants of the polynucleotides as per the present invention are provided for herein and is enabled by the unique ability of the present invention to allow for a plethora of drug cargoes via the in situ engineered EV production technology. The benefits of using self-amplifying RNA as the polynucleotide cargo are that, once the self-amplifying RNA is delivered to the tissues, multiple copies of the RNA are made, resulting in even more copies of the POI being made, by virtue of the amplification property of the RNA template. Importantly, the self-amplifying RNA replicon is not infectious and does not lyse cells, ensuring sustained protein expression. Amplification therefore results in very high RNA copy numbers, thereby achieving effective protein production at a much lower dose.

The terms “translation” and “expression” are used interchangeably herein and shall be understood to relate to and include the various steps resulting in a polypeptide being produced from a corresponding polynucleotide, including but not limited to (i) replication (e.g. DNA producing copies of itself with the aid of e.g. a DNA polymerase, which normally forms part of a group of factors called the replisome), (ii) transcription (production of RNA (such as a pre-mRNA) from a DNA template with the aid of e.g. RNA polymerase and other factors), (iii) processing of an RNA ((such pre-mRNA) via splicing, addition of 5′ cap and polyA tail, and other forms of RNA processing) into an mRNA, (iv) and translation of an mRNA into the corresponding protein with the help of the ribosomal machinery. Thus, the term “translation” as used in the present invention encompasses all the steps in converting the information in a polynucleotide to a corresponding protein, including the process of actual translation which converts an mRNA into a protein.

The terms “administration” and “administer” shall be understood to relate to different means of providing a composition to a subject, e.g. a patient. Administration may include providing a composition to a subject via various different routes of administration and also various different dosing and/or treatment regimens. A single dose of the compositions of the present invention may be sufficient in certain context, but multiple doses are envisaged in most diseases and conditions. Multiple doses may also be administered via multiple different routes, for instance a combination of intravenous and intrathecal or a combination of subcutaneous and intramuscular. Routes of administration contemplated herein include but are not limited administration via routes such as auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intra cisterna magnum, intro-colonic, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrarectal, intrasinal, intraspinal, intrasynovial, intratesticular, intrathecal, intrathoracic, intratubular, intratumour, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, the characteristics of the patient population, and/or the desired characteristics of the patient-derived genetically engineered EVs per se. Intravenous (IV), subcutaneous (SC), intrathecal (IT), intracerebroventricular (ICV), intra cisterna magnum (ICM), intraperitoneal (IP), and intramuscular (IM) are among the preferred routes of administration according to the present invention.

In a first aspect, the present invention relates to a composition comprising a delivery vector which comprises a polynucleotide cargo coding for a fusion protein, wherein translation of the polynucleotide cargo into the corresponding fusion protein results in the production of at least one EV comprising said fusion protein. As described above, the translation of the polynucleotide cargo by a cell in vivo results in the production of genetically engineered EVs comprising the fusion protein in question, which is surmised to be incorporated into the EV as part of the EV biogenesis process when it takes place in the cell in vivo. A polynucleotide cargo can be delivered to a target cell using a variety of viral or non-viral delivery vectors and the choice of vector will depend on numerous parameters, including desired target cell type and organ, size of the polynucleotide, whether the polynucleotide is comprised of DNA or RNA or both, the frequency of dosing (which can be reduced significantly by using a viral vector instead of a non-viral vector), the need to re-dose over time, and considerations related to expression levels, toxicity, immunogenicity and numerous other factors. In alternative embodiments, the polynucleotide cargo, for instance a modified mRNA, self-amplifying RNA or a plasmid DNA, can be delivered into target cells for in situ production of the fusion protein-carrying genetically engineered EVs without the use of a delivery vector in the conventional sense of the word. For instance, in some embodiments, the present invention relates to direct administration of the polynucleotide in merely a pharmaceutically acceptable carrier, for instance directly into a muscle tissue (IM), into the heart, into a solid tumour or any other cancerous tissue, or into the central nervous system including the brain, for instance via ICV, IT, or ICM administration of the polynucleotide. Even in the absence of e.g. an LNP or a polymer-based vector, the pharmaceutical composition in which the polynucleotide cargo is formulated will function as the delivery vector for all intents and purposes of the present invention. In addition to local administration into particular tissues, systemic administration of a polynucleotide is also contemplated herein. One particularly suitable approach may be hydrodynamic administration, which can be carried out intravenously to deliver the polynucleotide to cells of the liver or via intestinal administration, to allow for delivery of the polynucleotide to endothelial cells and other cells of the gastrointestinal system. As abovementioned, the formulation of a polynucleotide directly into a pharmaceutical composition (for instance directly in saline solution or any other physiologically suitable solution) is considered to be equivalent to the use of a composition comprising a non-viral delivery vector and the polynucleotide in question, as long as the key property of the polynucleotide being translated into engineered EVs carrying the fusion protein comprising the POI is maintained.

In a preferred embodiment, the composition comprises a non-viral vector. Non-viral vectors suitable for the present invention include lipid-based delivery vectors, such as a lipid nanoparticle, a liposome, a lipoplex, a lipidoid, a lipid emulsion, a cationic lipid, a zwitterionic lipid, or any other type of lipid based delivery vector. Furthermore, polymer-based delivery vectors are also suitable for the application of the present invention. Such polymer-based vectors include for instance polyplexes and polyamines. Other forms of delivery vectors include peptide-based vector, for instance cell-penetrating peptide (CPPs) delivery vectors (including CPPs that may form a polyplex with the cargo), or any other non-viral delivery vector suitable for the delivery of a polynucleotide cargo in vivo.

Lipid-containing delivery vectors in the form of nanoparticle compositions have proven very effective as transport vehicles into cells and/or intracellular compartments for a variety of different types of polynucleotides, notably DNA and messenger RNA (mRNA), including modified versions thereof. These lipid-based delivery vectors generally include one or more “cationic” and/or amino (ionizable) lipids, phospholipids including polyunsaturated lipids, structural lipids (e.g., sterols), and lipids containing polyethylene glycol (PEG lipids). Cationic and/or ionizable lipids include, for example, amine-containing lipids that are easily protonated to turn them cationic. By employing lipid or lipid-like materials (lipidoids), various delivery vectors can be prepared, e.g. liposomes, lipid nanoparticles (LNPs), lipid emulsions, lipid implants, etc. For example, N-[1-(2, 3-dioleoyloxy)propyl]-N,N,N-trimethylammonium chloride (DOTMA), 1,2-dioleoyloxy-3-trimethylammonium propane chloride (DOTAP), 1,2-dioleoyl-sn-glycero-3-phosphoethanolamine (DOPE), as classical cationic lipids, has been extensively used to deliver DNA and mRNA and DOTMA, DOTAP, DOPE and cholesterol has been used to deliver mRNA to dendritic cells, macrophages, and lung endothelial cells, etc. Other lipid based compounds which can be used to form lipidoids, LNPs and other types of lipid based delivery vectors include DlinDMA, Dlin-MC3-DMA, Dlin-MC3-DMA with backbone modifications including with ester and alkyne in the lipid tail, C12-200, cKK-E12, 5A2-SC8, 7C1 and 1,3,5-triazinane-2,4,6-trione (TNT) derivatives, MC3, XTC2, etc. Based on for instance these lipids or lipidoids, effective highly effective mRNA delivery and fusion protein expression can be achieved by adjusting the molar ratio of key lipids to helper lipids, PEG-lipids and cholesterol, changing the helper lipids or PEG-lipids, adding another components (e.g. protamine).

Liposomes is one type of non-viral lipid-based delivery vector suitable in the context of the present invention to form part of the composition together with the polynucleotide. The liposome-incorporated polynucleotide may be completely or partially located in the interior space of the liposome, within the bilayer membrane of the liposome, or associated with the exterior surface of the liposome membrane. While liposomes can facilitate introduction of nucleic acids into target cells, the addition of polycations (e.g., poly L-lysine and protamine), as a copolymer can facilitate, and in some instances markedly enhance the delivery efficiency of several types of cationic liposomes. In a preferred embodiment of the present invention, the lipid based delivery vector is formulated as a lipid nanoparticle. As used herein, the phrase “lipid nanoparticle” refers to a delivery vector comprising one or more lipids (e.g., cationic lipids, non-cationic lipids, and PEG-modified lipids). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatine, alginate, collagen, chitosan, cyclodextrins, dendrimers and polyethyleneimine. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. Other suitable lipids include (15Z, 18Z)—N,N-dimethyl-6-(9Z, 12Z)-octadeca-9, 12-dien-1-yl)tetracosa-15,18-dien-1-amine (HGT5000), (15Z, 18Z)—N,N-dimethyl-6-((9Z, 12Z)-octadeca-9, 12-dien-1-yl)tetracosa-4,15,18-trien-1-amine (HGT5001), and (15Z,18Z)—N,N-dimethyl-6-((9Z, 12Z)-octadeca-9, 12-dien-1-yl)tetracosa-5,15, 18-trien-1-amine (HGT5002). In some embodiments, the cationic lipid N-[1-(2,3-dioleyloxy)propyl]-N,N,N-trimethylammonium chloride or “DOTMA” is used. DOTMA can be formulated alone or can be combined with the neutral lipid, dioleoylphosphatidyl-ethanolamine or “DOPE” or other cationic or non-cationic lipids into a liposome or a lipid nanoparticle. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA”, 1,2-Dioleoyl-3-Dimethylammonium-Propane or “DODAP”, 1,2-Dioleoyl-3-Trimethylammonium-Propane or “DOTAP”. Other contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-I-(cis,cis-9′, I-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N, N′-Dilinoleylcarbamyl-3-dimethylaminopropane or “DLincarbDAP”, I,2-Dilinoleoylcarbamyl-3-dimethylaminopropane or “DLinCDAP”, 2,2-dilinoleyl-4-dimethylaminomethyl-[1,3]-dioxolane or “DLin-DMA”, 2,2-dilinoleyl-4-dimethylaminoethyl-[I,3]-dioxolane or “DLin-K-XTC2-DMA”, and 2-(2,2-di((9Z,12Z)-octadeca-9,I2-dien-1-yl)-1,3-dioxolan-4-yl)-N,N-dimethylethanamine (DLin-KC2-DMA)), or mixtures thereof. The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Choi (N,N-dimethyl-N-ethylcarboxamidocholesterol), I,4-bis(3-N-oleylamino-propyl)piperazine. Other suitable cationic lipids include dialkylamino-based, imidazole-based, and guanidinium-based lipids. For example, certain embodiments are directed to a composition comprising one or more imidazole-based cationic lipids, for example, the imidazole cholesterol ester or “ICE” lipid.

Similarly, certain embodiments are directed to lipid nanoparticles comprising the HGT4003 cationic lipid 2-((2,3-Bis((9Z,12Z)-octadeca-9,12-dien-1-yloxy)propyl)disulfanyl)-N,N-dimethylethanamine. In other embodiments the compositions and methods described herein are directed to lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005). The use of polyethylene glycol (PEG)-modified phospholipids and derivatized lipids such as derivatized cerarmides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipids together which comprise the delivery vector (e.g., the lipid nanoparticle). Contemplated PEG-modified lipids include, but is not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-polynucleotide composition to the target cell, or they may be selected to rapidly exchange out of the formulation in vivo. Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the lipid based delivery vector. In alternative embodiments, the non-viral lipid based delivery vector may also use non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, l-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the delivery vector. Preferably, a lipid nanoparticle is prepared by combining multiple lipid and/or polymer components. For example, the delivery vector may be prepared using CI 2-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the polynucleotide (typically a modified mRNA) to be delivered. Additional considerations include, for example, the saturation of the alkyl chain, as well as the size, charge, pH, pKa, fusogenicity and toxicity of the selected lipid(s). Thus, the molar ratios may be adjusted accordingly. For example, in embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%. In certain embodiments, the lipid nanoparticles of the invention comprise at least one of the following cationic lipids: C12-200, DLin-KC2-DMA, DODAP, HGT4003, ICE, HGT5000, or HGT5001. In embodiments, the delivery vector comprises cholesterol and/or a PEG-modified lipid. In some embodiments, the delivery vector comprises DMG-PEG2K and in some embodiments the delivery vector comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, DMG-PEG2K.

In preferred embodiments, the lipid based delivery vectors of the present invention uses PEG lipids selected from the non-limiting group consisting of PEG-modified ceramides, PEG-modified dialkylamines, PEG-modified diacylglycerols, PEG-modified phosphatidylethanolamines, PEG-modified phosphatidic acids, and PEG-modified dialkylglycerols, whereas structural lipids may include cholesterol fecosterol, sitosterol, ergosterol, ursolic acid, or alpha-tocopherol. The lipid component may include one or more phospholipids, such as one or more (poly)unsaturated lipids. In general, such lipids may include a phospholipid moiety and one or more fatty acid moieties.

As abovementioned, polymer-based non-viral delivery vectors are also contemplated in the present invention as delivery vectors for polynucleotides, in particular mRNA and plasmid DNA. A polymer may be biodegradable and/or biocompatible and may be selected from, but not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D, L-lactide), poly(D, L-lactide-co-PPO-co-D, L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as polyvinyl acetate), polyvinyl halides such as polyvinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate.

In another embodiment of the present invention, the delivery vector of the composition is a viral delivery vector, i.e. a virus which is modified to carry the polynucleotide cargo. Suitable viral vectors include adenovirus, adeno-associated virus (AAV), lentivirus, vesicular stomatitis virus, vaccinia virus, alphavirus, flavivirus, rotavirus, retrovirus, herpes simplex virus, respiratory syncytial virus, virus-like particle (VLP), or any other viral delivery vector suitable for a polynucleotide cargo. In a preferred embodiment, the viral delivery vector of the present invention is an AAV or a lentivirus. AAVs exist in numerous natural serotypes, including AAV1, AAV2, AAV3, AAV4, AAV5, AAV6, AAV7, AAV8, AAV9, all of which are suitable viral carriers for the polynucleotides of the present invention. Also, various recombinant and modified AAV serotypes exist, and also all of these variants are suitable for application in the context of the present invention. The selection of AAV serotype is primarily driven by considerations related to from which target cell one desires to transduce with the polynucleotide cargo. For instance, AAV7, AAV8, and AAV9 are highly efficient at transducing the liver, which is a preferred embodiment of the present invention as it results in production of the engineered EVs comprising the fusion protein as a result of transcription and translation of the polynucleotide cargo. AAV1, AAV6, AAV7, AAV8, and AAV9 are also suitable viral vector as they can target muscle cells for production of engineered EVs comprising the fusion protein in question. Targeting of central nervous system cells is best achieved using AAV1, AAV2, AAV4, AAV5, AAV8, and AAV9, which can result in production of the engineered EVs comprising the fusion protein comprising the POI for therapeutic applications in the central nervous system. Lentiviruses are another suitable viral delivery vector for the polynucleotides of the present invention and have the advantage of being able to carry larger transgenes (i.e. the polynucleotides) than AAVs.

In a preferred embodiment, the delivery vector of the present invention is a virus like particle (VLP). VLPs are self-assembled polypeptide structures with sizes ranging 20-800 nm that mimic the organization and configuration of native viruses, but lack the viral genome and therefore have the potential to produce safer and cheaper drug delivery vesicles. A VLP is a self-assembled particle formed from at least one component that assembles spontaneously; the component may be a polypeptide or a non-peptide compound. VLP may be composed of one or more peptides, the one or more peptides may be the same or different polypeptide. The polypeptide may be a viral structural polypeptide, therefore, the VLP may be similar to virus particles. The viral structural polypeptide may be a naturally occurring viral polypeptide or a modified polypeptide thereof. The viral polypeptide may be a naturally-occurring viral structural polypeptide including a capsid and envelope protein. For example, the envelope protein may include at least one protein selected from the group consisting of E3, E2, 6K, and E1 and/or the capsid protein may be at least one of VP1, VP2, VP3 or VP4. The viral protein making up the VLP may be derived from a wide variety of virus families including but not limited to Hepatitis C, alpha viruses, Parvoviridae (e.g. adeno-associated virus), Retroviridae (e.g. HIV), Flaviviridae (e.g. Hepatitis C virus), Paramyxoviridae (e.g. Nipah) and bacteriophages (e.g. Qβ, AP205). VLPs can be produced in multiple cell culture systems including bacteria, mammalian cell lines, insect cell lines, yeast and plant cells.

In some embodiments, viral and non-viral vectors may advantageously be combined into hybrid vectors, comprising for instance a virus like AAV and a lipid nanoparticle or similar, or viral components combined with any type of non-viral delivery vector. Generally, both viral and non-viral delivery technologies are well-known and well-understood for the delivery of polynucleotides to target cells and as such the skilled person is well acquainted with the manufacturing process for these vectors, including how to leverage contract manufacturing services from commercial entities to carry out GMP manufacture.

Depending on the desired delivery vector, the polynucleotide cargo of the compositions of the present invention may comprise RNA or DNA or both RNA and DNA, which may both be either single stranded or double stranded. For example, if the composition utilizes a viral delivery vector in the form of an AAV virus, then the polynucleotide needs to be a single-stranded DNA which is the format of the AAV genome, whereas if the viral delivery vector is a retrovirus such as a lentivirus, then the polynucleotide is an single stranded RNA molecule. When the composition of the present invention is utilizing a viral delivery vector the production from a given target cell in vivo of engineered EVs is sustained over extended time periods, as a result of essentially stable transduction of the EV producing cell. For instance, for AAV viral vectors, the expression of a transgene can last for decades, especially in non-dividing tissues. However, if short term, transient expression and translation of the fusion protein (for delivery of a therapeutic POI) into the engineered EVs produced by the cells into which the composition delivers the polynucleotide is desired, then non-viral vectors such as LNPs, liposomes, polymer-based vectors or CPPs may be preferable. Single stranded linear or circular mRNA or DNA (e.g. plasmid DNA) is a particularly advantageous embodiment in combination with an LNP or liposome-based delivery vector or with a CPP vector. mRNA polynucleotides are preferably modified to increase stability, reduce immunogenicity and improve PK/PD properties more generally. The polynucleotide cargo of the present invention may thus be selected from the group which consists of, but which is not limited to, linear mRNA, circular mRNA, linear DNA, circular DNA, plasmid DNA, linear RNA, circular RNA, self-amplifying RNA or DNA, viral genome, or modified versions of any of the above, as well as any other suitable polynucleotide cargo.

In specific embodiments the delivery vector is an LNP and the cargo nucleic acid is an mRNA; the delivery vector is an LNP and the cargo nucleic acid is a plasmid; the delivery vector is a VLP and the cargo nucleic acid is an mRNA or the delivery vector is an LNP and the cargo nucleic acid is a plasmid.

In a preferred embodiment of the present invention, the fusion protein encoded for by the polynucleotide cargo of the composition comprises at least one EV polypeptide and at least one protein of interest (POI). An EV polypeptide is essentially any protein, region, domain, motif, or sequence or stretch of amino acids that is capable of transporting the fusion protein into an EV produced by a given EV-producing cell. Without limiting the generality of the term EV polypeptide, preferred EV polypeptides comprised in the fusion proteins as per the present invention may be transmembrane EV polypeptides; specific preferred EV polypeptides can be selected from the group consisting of the following non-limiting examples: CD9, CD53, CD63, CD81, CD54, CD50, FLOT1, FLOT2, CD49d, CD71, CD133, CD138, CD235a, AAAT, AT1B3, AT2B4, ALIX, Annexin, BASI, BASP1, BSG, Syntenin-1, Syntenin-2, Lamp2, Lamp2a, Lamp2b, TSN1, TSN3, TSN4, TSN5 TSN6, TSN7, TSPAN8, TSN31, TSN10, TSN11, TSN12, TSN13, TSN14, TSN15, TSN16, TSN17, TSN18, TSN19, TSN2, TSN4, TSN9, TSN32, TSN33, TN FR, TfR1, syndecan-1, syndecan-2, syndecan-3, syndecan-4, CD37, CD82, CD151, CD224, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, CLIC1, CLIC4, interleukin receptors, CD2, CD3 epsilon, CD3 zeta, CD13, CD18, CD19, CD30, CD34, CD36, CD40, CD40L, CD44, CD45, CD45RA, CD47, CD53, CD86, CD110, CD111, CD115, CD117, CD125, CD135, CD184, CD200, CD279, CD273, CD 274, CD362, COL6A1, AGRN, EGFR, FPRP, GAPDH, GLUR2, GLUR3, GP130, GPI anchor proteins, GTR1, HLAA, HLA-DM, HSPG2, ITA3, Lactadherin, L1CAM, LAMB1, LAMC1, LIMP2, MYOF, ARRDC1, ATP2B2, ATP2B3, ATP2B4, BSG, IGSF2, IGSF3, IGSF8, ITGB1, ITGA4, ATP1A2, ATP1A3, ATP1A4, ITGA4, SLC3A2, ATP transporters, ATP1A1, ATP1B3, ATP2B1, LFA-1, LGALS3BP, Mac-1 alpha, Mac-1 beta, MFGE8, a member of the myristoylated alanine rich Protein Kinase C substrate (MARCKS) protein family such as MARCKSL1, matrix metalloproteinase-14 (MMP14), PTGFRN, BASP1, MARCKS, MARCKSL1, PRPH2, ROM1, SLIT2, SLC3A2, SSEA4, STX3, TCRA, TCRB, TCRD, TCRG, TFR1, UPK1A, UPK1B, VTI1A, VTI1B, and any other EV polypeptide, and any combinations, derivatives, domains, variants, mutants, or regions thereof. Mutations, truncations, linkers or additions may be introduced into the wild type sequence of the EV polypeptide to alter its function, for instance a preferred mutant according to the invention is a mutation of the tetraspanin CD63 which replaces the tyrosine in position 235 with alanine (denoted CD63/Y235A). The use of EV proteins has the effect of driving loading of the fusion protein into EVs, such that not only is the POI located in the EV and subsequently secreted by the EV-producing cell but the production of EVs comprising the fusion protein also increased by virtue of the pressure exerted on the EV-producing cell to express and translate the delivered polynucleotide cargo. Particularly advantageous EV polypeptides include CD63, CD81, CD9, CD82, CD44, CD47, CD55, LAMP2B, LIMP2, ICAMs, integrins, ARRDC1, syndecan, syntenin, PTGFRN, BASP1, MARCKS, MARCKSL1, TfR, and Alix, as well as derivatives, domains, variants, mutants, or regions thereof. In some embodiments, the EV polypeptide may be combined with transmembrane domains from various cytokine receptors, for instance TNFR and gp130, in order to enhance the loading of the fusion protein into the genetically engineered EVs.

The POI comprised in the fusion protein as per the present invention is normally a pharmacologically active agent, such as an enzyme, a receptor, a nucleic acid-interacting protein such as a tumour suppressor or a transcription factor, or any other suitable protein which can mediate a pharmacological effect in the context of a given disease. The POI may be selected from the group consisting of non-limiting examples such as an enzyme, a transporter protein, a transmembrane protein, a structural protein, a transcription factor protein, a tumour suppressor protein, a nuclear protein, a receptor protein, a protein-binding protein, a nucleic acid-binding protein, a nuclease, a recombinase, a chaperone protein, a translation regulatory protein, a transcription regular protein, a toxin protein, a binding protein, a molecular carrier protein, an immune system protein, a metabolic protein, a signalling protein, nucleic acid-binding proteins, nucleases, recombinases, and protein-binding proteins or any other type of protein. In preferred embodiments, the POI is a therapeutic protein selected from the group consisting of enzymes, transporters, chaperones, transmembrane proteins, structural proteins, nucleic acid-binding proteins such as tumour suppressors, transcription factors, nucleases (for instance Cas9, Cas6, meganucleases, etc.), recombinases, and protein-binding proteins.

The fusion proteins of the present invention may in advantageous embodiments further comprise various domains intended to endow the engineered EVs with additional properties to enhance their pharmacological, pharmacokinetic, or biodistribution behaviour in vivo. For example, the fusion protein can be design to contain a targeting domain in the form of, for instance, a targeting peptide, a single-chain antibody derivative (such as a VHH, a VNAR, an alphabody, an affibody, a centyrin, heavy chain only antibodies, a humabody, or a nanobody) or any other form of targeting entity. A non-limiting example of a fusion protein with a targeting moiety fused to it is the fusion between a VHH targeting the transferrin receptor on the blood-brain-barrier and the EV polypeptide Lamp2B and a given POI, preferably one which has pharmacological activity in the central nervous system. Further, targeting moieties may be used to target the EVs to cells, subcellular locations, tissues, organs or other bodily compartments. Organs and cell types that may be targeted include: the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells. Targeting can be achieved by a variety of means, for instance the use of targeting peptides. Such targeting peptides may be anywhere from a few amino acids in length to several 100s of amino acids in length, e.g. anywhere in the interval of 3-200 amino acids, 3-100 amino acids, 5-30 amino acids 5-25 amino acids, e.g. 7 amino acids, 12 amino acids, 20 amino acids, etc. Targeting peptides of the present invention may also include full length proteins such as receptors, receptor ligands, etc. Exemplary targeting moieties include brain targeting moieties such as RVG, NGF, melanotransferrin and the scFv FC5. Peptide and muscle targeting include moieties such as Muscle Specific Peptide (MSP).

In another advantageous embodiment, the fusion protein further comprises at least one cleavable domain, to enable release of the POI from the fusion protein. Non-limiting examples of cleavable domains include domains which has protease cleavage sites in the amino acid sequence or cis-cleaving domains which are self-cleaving. Suitable release domains according to the present invention may be cis-cleaving sequences such as inteins, light induced monomeric or dimeric release domains such as Kaede, KikGR, EosFP, tdEosFP, mEos2, PSmOrange, the GFP-like Dendra proteins Dendra and Dendra2, CRY2-CIBN, etc. Alternatively, nuclear localization signal (NLS)—nuclear localization signal-binding protein (NLSBP) (NLS-NLSBP) release system may be employed. Protease cleavage sites may also be incorporated into the fusion proteins for protease-triggered release, etc., depending on the desired functionality of the fusion polypeptide. In the case of the POI binding to and transporting a nucleic acid cargo (e.g. an mRNA) into the EV, specific nucleic acid-cleaving domains may be included. Non-limiting examples of nucleic acid cleaving domains include endonucleases such as Cas6, Cas13, engineered PUF nucleases, site specific RNA nucleases etc. Preferred embodiments of self-cleaving domains include cis-cleaving domains such as inteins. Self-cleaving domains are particular advantageous when combined with enzymes that need to be soluble in a target cell compartment, for instance the cytoplasm, the mitochondria, the nucleus, and/or the endo-lysosomal system. Non-limiting examples of such fusion proteins include the EV polypeptides CD63, CD9, CD81, Lamp, PTGFRN, MARCKS, MARCKSL1, BASP1, a self-cleaving intein, and a POI such as a lysosomal storage disorder (LSD) enzyme, a urea cycle enzymes, or any enzyme that is disrupted or mutated in an inborn error of metabolism (non-limiting examples of such enzymes include N-acetylglutamate synthase, carbamoyl phosphate synthetase, ornithine transcarbamoylase, carbamyl phosphate synthetase, argininosuccinic acid synthase, argininosuccinate synthetase, argininosuccinic acid lyase (also known as argininosuccinate lyase), arginase, mitochondrial ornithine transporter, ornithine translocase, citrin, phenylalanine dehydroxylase, cystathionine beta synthase, methylmalonyl CoA mutase, methylmalonyl CoA epimerase, imiglucerase, alpha-galactosidase, alpha-L-iduronidase, iduronate-2-sulfatase, idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin 1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, and hexoaminidase) and/or any other intracellular enzyme or protein.

Further, in order to extend the circulation time and specifically plasma half-life of the engineered EVs even further, the fusion protein may further comprise a polypeptide domain which binds to a suitable plasma and/or blood protein. An exemplary embodiment of this is the inclusion in the fusion protein of an albumin-binding polypeptide, in order for the engineered EVs produced by the cell and secreted into the extracellular milieu to bind to serum albumin (human serum albumin being the human version). The terms albumin-binding polypeptide or albumin-binding domain (ABD) are used interchangeably herein and shall be understood to relate to any protein, peptide, antibody or nanobody, or fragment or domain thereof capable of binding to albumin. ABDs may be derived from any species, preferably the ABP has specific binding affinity for human serum albumin. Commonly known ABDs are antibodies or nanobodies that are raised against albumin or ABDs derived from PAB protein from Peptostreptococcus magnus and protein G from group C and G streptococci, both of which bind to albumin with high affinity. ABDs are often small three-helical protein domains found in various surface proteins often expressed for instance by gram-positive bacteria. Albumin-binding domains found in nature may be engineered by specific mutagenesis to achieve a broader specificity for different albumin, an increased stability, lower immunogenicity or an improved binding affinity. The ABDs comprised in the fusion protein of the present invention may also be an antibody, scFv, nanobody, heavy chain antibody (hcAb), single domain antibody (sdAb) such as VHH or VNAR, or a fragment thereof which is capable of binding to albumin. sdAbs and antibody fragments are particularly preferred due to their small size which allows for other additional domains to be introduced into the fusion protein and simple construct design and expression/translation. In order to mediate albumin-binding, ABDs according the present invention are engineered into the polynucleotide and hence the resultant fusion protein to be present on the surface of the EV so that they are able to bind to albumin found primarily in the circulatory systems. The ABD may be presented on the surface of the EV in any number of ways provided that the ABD is exposed on the outer surface of the EV such that it is capable of binding albumin.

Importantly, the compositions of the present invention allow for viral and non-viral delivery of polynucleotide constructs into EV-producing cells in vivo, leading to production of EVs having the POI incorporated therein and thus ultimately EV-mediated delivery of the POI into various target tissues. This approach to in situ or endogenous drug delivery represents a completely novel approach to EV therapeutics and endows the engineered EVs with both a pharmacologically active agent (in the form of the POI, or in the form of an agent that the POI binds to and transports into the EV) and the characteristics of the EVs of the subject itself, allowing for essentially autologous EV therapy without the need to harvest EVs from the patient. The ability to utilise a composition comprising a polynucleotide coding for the fusion protein comprising the POI means that not only is the resultant patient-derived EVs inherently well tolerated but they also exhibit superior PK/PD properties and is significantly less costly to produce, with cost of goods comparable to for instance, mRNA therapeutics, when a modified mRNA is used as the polynucleotide cargo with a non-viral vector in the composition. In preferred embodiments of the present invention, the EVs comprising the fusion proteins produced from the polynucleotides comprised in the compositions are patient cell-specific EVs, preferably derived from cell types such as liver cells, muscle cells and/or cells of the central nervous system or the brain. Thus, preferred alternatives include but are not limited to genetically engineered patient-derived EVs which preferably are genetically engineered EVs derived from the liver cell of a patient (i.e. genetically engineered patient liver cell-derived EVs), from a CNS or a brain cell of a patient (i.e. genetically engineered patient CNS or brain cell-derived EVs) or from a muscle cell of a patient (i.e. genetically engineered patient muscle cell-derived EVs).

In a preferred embodiment, the composition of the present invention comprises a delivery vector that is a lipid nanoparticle and a polynucleotide cargo which is either an mRNA, self-amplifying RNA or a plasmid DNA. Plasmid DNA (pDNA) is advantageous because of its ability to be delivered episomally into target cells, resulting in long-term expression of the corresponding fusion protein which results in the production of engineered EVs comprising the fusion protein. When the polynucleotide is pDNA, which is composed of double-stranded DNA, the polynucleotide needs to be converted into the corresponding fusion protein via the conventional steps of the central dogma of molecular biology, namely transcription of DNA into RNA followed by various processing steps and translation of the RNA (i.e. the mRNA) into the resultant fusion protein. For clarity, the terms translation and expression (which are used interchangeably herein) as used in the context of the present invention shall be understood to comprise all the required steps for converting a polynucleotide sequence (which may comprise DNA, RNA, or a combination of the two) into an amino acid sequence, including transcription of DNA into RNA, reverse transcription of a RNA into DNA (as carried out by retroviruses such as lentivirus), processing of RNA into mRNA, translation of mRNA into a fusion protein, and any other intermediate steps or processes. In addition to pDNA, other forms of polynucleotides exist that have sustained ability to translate the fusion protein and thereby create engineered EVs that comprise the fusion protein. Such other forms of long-lasting polynucleotides include self-replicating polynucleotides such as self-amplifying RNA, viral genomes, circular mRNA, episomes, capsid-free AAV genomes, and other forms of polynucleotides.

As abovementioned, a preferred embodiment of the present invention is a lipid nanoparticle delivery vector and an mRNA as the polynucleotide. An mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine. A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component. A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein. An mRNA may include a 5′ untranslated region, a 3′ untranslated region, and/or a coding or translating sequence, which is translated to create the fusion protein of the present invention. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In some embodiments, an mRNA may include a 5′ cap structure, a chain terminating nucleotide, a stem loop, a polyA sequence, and/or a polyadenylation signal. A cap structure or cap species is a compound including two nucleoside moieties joined by a linker which caps the mRNA at its 5′ end, and which may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, iri27′03′GpppG, iri27′03′GppppG, iri27′02′GppppG, m7Gpppm7G, m73′dGpppG, iri27′03′GpppG, iri27′03′GppppG, and m27 02′GppppG. An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. An mRNA may instead or additionally include a stem loop, such as a histone stem loop. A stem loop may include 1, 2, 3, 4, 5, 6, 7, 8, 9 or more nucleotide base pairs. For example, a stem loop may include 4, 5, 6, 7, 8, 9 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. The modified mRNA of the present invention may comprise in addition to the coding region (which codes for the fusion protein and which may be codon-optimized) one or more of a stem loop, a chain terminating nucleoside, miRNA binding sites, a polyA sequence, a polyadenylation signal, 3′ and/or 5′ untranslated regions (3′ UTRs and/or 5′ UTRs) and/or a 5′ cap structure. As abovementioned, various nucleotide modifications are preferably incorporated into the mRNA to modify it for increased translation, reduced immunogenicity, and increased stability. Suitable modified nucleotides include but are not limited to N1-methyladenosine (m1A), N6-methyladenosine (m6A), 5-methylcytidine (m5C), 5-methyluridine (m5U), 2-thiouridine (s2U), 5-methoxyuridine (5moU), pseudouridine (ψ), N1-methylpseudouridine (m1ψp). Among these mRNA modifications, m5C and ψ are the most preferred as they reduce the immunogenicity of mRNA as well as increase the translation efficiency in vivo. In preferred embodiments of the present invention, the composition herein comprises a non-viral delivery vector such as an LNP or a liposome comprising a modified mRNA as the polynucleotide cargo, wherein the mRNA is modified with at least 50% m5C and 50% ψ or m1ψ, preferably at least 75% m5C and 75% ψ or m1ψ, and even more preferably 90% m5C and 90% ψ or m1ψ, or even more preferably 100% modification using m5C and ψ or m1ψ. Such modified mRNAs polynucleotide preferably code for fusion proteins comprising (i) an EV polypeptide such as a tetraspanin (for instance CD63, CD81, CD9), PTGFRN, or Lamp2, (ii) a self-cleaving polypeptide domain, for instance a cis-cleaving domain, such as an intein, and (iii) a POI in the form of an enzyme which is deficient in a disease selected from the inborn errors of metabolism, e.g. PAH, ASL, ASS, GAA, GLA, etc. In another preferred embodiments, the components (i), (ii) and (iii) can be further combined with (iv) a targeting entity expressed on the external surface of the engineered EV, thereby directing delivery to a preferred target cell and/or tissue, and (v) a polypeptide domain which binds to serum albumin, the further extend the already long half-life of the engineered EV comprising the POI.

In a second aspect, the present invention relates to a pharmaceutical composition comprising the compositions as described herein (i.e. the composition comprising a delivery vector and the polynucleotide encoding for the fusion protein which when the polynucleotide is expressed leads to translation of the fusion protein and generation of EVs comprising the fusion protein). The compositions of the present invention are already suitable for pharmaceutical purposes but may in a further step be formulated in a pharmaceutically acceptable formulation. For example, the compositions of the present invention may be formulated with one or more pharmaceutically acceptable excipients or accessory ingredients such as, but not limited to, one or more solvents such as aqueous solvents including saline solution, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, colouring agents, and coating agents may also be included. Exemplary excipients include excipients intended to reduce degradation or loss of activity, for instance proteins such as human serum albumin, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca2+, Zn2+ and EDTA, cyclodextrin-based such as hydroxypropyl ß-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol. The term composition and pharmaceutical composition are used interchangeably herein and when one of said compositions is contemplated the other of said compositions is contemplated too.

It is envisaged that the pharmaceutical compositions of the present invention may be formulated as an intravenous formulation, parenteral formulation or any type of modified release formulation; an oral formulation (tablet, capsule or liquid) is also possible. In a particular embodiment, the pharmaceutical compositions are in liquid form. The dosage regime will depend on the cargo being delivered, the disease to be treated and any additional therapies being administered, which will be readily determined by the skilled physician. It is envisaged that the compositions of the present invention will be administered multiple times, i.e. more than 1 time, but normally more than 2 times, or potentially for chronic, long-term treatment (i.e. administered tens to hundreds to thousands of times). i.e. as part of a chronic treatment regimen. Dosage amounts may depend on the vector and/or cargo, but will be readily determined by the skilled physician. Illustrative examples include a range of 0.001-10 mg/kg (e.g. 0.1-5 mg/kg) for LNPs, a range of 1×10⁹-1×10¹⁵ vg/kg (e.g. 1×10¹¹-1×10¹³) for AAVs and a range of 0.001-10 mg/kg (e.g. 0.1-5 mg/kg) for saRNA.

In a third aspect, the present invention relates to the composition as per the present invention for use in medicine. Suitable formulations, routes of administration, dosage amounts, regimes etc. are as described for the pharmaceutical compositions disclosed herein. More specifically, the compositions herein may be for use in the treatment and/or prophylaxis of essentially any disease, disorder, condition, or ailment, preferably selected from the group consisting of genetic diseases, hereditary diseases (including both genetic diseases and non-genetic hereditary diseases), lysosomal storage disorders, inborn errors of metabolism, urea cycle disorders, neuromuscular diseases, neurodegenerative diseases, cancer, autoimmune diseases, cardiovascular diseases, central nervous system diseases, infectious diseases, and inflammatory diseases. Numerous diseases resulting from gene defects are particularly suitable for treatment with the compositions and the resulting engineered EVs of the present invention, namely such diseases wherein the replacement of a given protein (that is missing or defective as a result of a genetic defect) can be achieved by delivering the fusion protein which comprises a POI which corresponds to the protein that should have been coded for by the defective gene. Such engineered EV-based replacement therapies can be seen as essentially EV-mediated protein replacement therapies and have the unique advantage of being able to deliver the POI not only to the extracellular environment but also into the intracellular (including lysosomal) and/or membrane environment, by virtue of the POI being present in the engineered EVs secreted by cells of the subject with the gene defect (i.e. a patient suffering for instance from an inborn error of metabolism, such as PKU, ASA, MMA, OTC, NPC, Pompe disease, Fabry disease, Gaucher's disease, etc.). Treatment can include the amelioration of the disease, disorder, condition or ailment and/or an improvement in symptoms. Prophylaxis can include partial or full prevention of the disease, disorder, condition or ailment. Any adult or child human patient population may be considered for treatment or prophylaxis.

In a further embodiment, the present invention relates to the compositions herein for use in a method of treatment of disease, wherein the method comprises to administer the composition to a target cell which is capable of producing and secreting EVs comprising the fusion protein, as a result of the translation of the polynucleotide cargo into the corresponding fusion protein. As noted above, the translation of a polynucleotide into the fusion protein (which is loaded into engineered EVs secreted by the target cell for the delivery of the composition) may include various steps preceding the actual translation of an mRNA into a protein, for instance reverse transcription, transcription, splicing and other forms of RNA processing, and, in the case of self-replicating polynucleotide cargoes, also replication. Importantly, the administering of the composition of the present invention to target cells of a patient results in the target cells of the patient producing patient-derived engineered EVs which comprise the fusion protein as a result of translation of the polynucleotide cargo, meaning that the fusion protein with the POI is present in EVs which are then secreted either locally and/or systemically in the body of the patient. The production of genetically engineered EVs comprising the fusion protein comprising the POI means that the pharmacological activity mediated by the POI (either the POI itself or any other agent with which the POI interacts) is not only limited to the cell which is the target of the compositions of the present invention, but that the natural delivery capabilities of EVs is harnessed for delivery of the POI in question. Critically, the genetically engineered EVs comprising the fusion protein produced from expression of the polynucleotide have a considerably extended circulation half-life as compared to ex vivo (i.e. in vitro) produced EVs. This is surmised to be the result of the homologous nature of the patient-derived EVs but it is likely also related to the distinct corona of host factors that are surmised to coat and/or associate with the genetically engineered patient-derived EV when produced and secreted in situ in the body of the patient. Ex vivo-manufactured genetically engineered EVs lack this important attribute and hence exhibit a different, less advantageous biodistribution/pharmacokinetics profile in vivo.

Importantly, from a cost-of-goods (COGs) perspective, the methods of treatment as per the present invention does not require scaling up manufacturing of genetically engineered EVs in vitro but merely require regulatory compliant (i.e. GMP) manufacturing of a suitable polynucleotide cargo molecule in a suitable delivery vector, for instance manufacturing of modified mRNA loaded into lipid nanoparticles. This means that COGs can be kept lower than what would have been the case with conventional EV manufacture and it also means that repeated administration of the composition is not only pharmacologically and pharmaceutically advantageous but also technically and commercially feasible. Repeated administration may be to the same target cell in the same target organ or may be focused on other target cells in other target organs, depending on the desired PK/PD outcome.

As the pharmacological (i.e. therapeutic) activity of the POI is mediated by EV delivery, it is important to stimulate significant EV production, preferably over extended time periods. Certain target organs are preferred as they result in high EV production. In advantageous embodiments, the compositions of the present invention are delivered as part of the method of treatment of disease to target organs such as the liver, the spleen, the lungs, muscle tissues, tissues of the central nervous system, the bone marrow, and/or any other tissue capable of secreting EVs at a high rate preferably over extended time periods. The liver is the preferred target organ for the compositions of the present invention, as the liver and especially hepatocytes and/or macrophages (such as Kupffer cells) of the liver can function as “in situ bioreactors” for secretion of the genetically engineered EVs comprising the fusion protein comprising the POI into the systemic circulation, thereby mediating body-wide pharmacological activity, for instance in the form of engineered EV-mediated protein replacement therapies for genetic diseases (e.g. urea cycle disorders, lysosomal storage disorders, or other inborn errors of metabolism).

In another aspect, the present invention relates to a method of manufacturing the compositions herein. The manufacturing methods typically comprise the steps of (i) providing a suitable polynucleotide cargo and (ii) incorporating the polynucleotide cargo into the chosen delivery vector. As abovementioned, certain compositions are preferred, notably modified mRNA which is incorporated into a lipid-based delivery vector such as an LNP or a liposome. The method for manufacturing an mRNA-containing composition may comprise the steps of (i) providing an in vitro transcribed (IVT) mRNA polynucleotide cargo with suitable nucleotide modifications and suitable components of the polynucleotide to support high translation (as described in detail above, e.g. UTRs, 5′ cap, poly(A) tail, etc.), and (ii) formulating the IVT mRNA polynucleotide cargo in a suitable lipid-based delivery vector such as an LNP, a lipidoid, a lipoplex, a liposome, or a lipid emulsion. The skilled person is well aware of for instance the best LNP formulation to use depending on factors such as length of the modified mRNA polynucleotide, modification patterns, secondary structure, and target cell type(s).

In yet another aspect, the present invention relates to a method of producing at least one EV comprising a fusion protein comprising an EV polypeptide and a POI in a mammalian cell, the method comprising putting the mammalian cell in contact with a composition as described herein, wherein the mammalian cell is capable of translating the polynucleotide cargo into the corresponding fusion protein resulting in the production of mammalian cell-derived EVs comprising the fusion protein. As abovementioned, the mammalian cell may be any cell of the body of a mammal, for instance a liver cell such as a hepatocyte or a liver macrophage (e.g. a Kupffer cell). Various other cells and cell types in other organs than the liver may also function as the “in situ bioreactors” which are important for the method of producing the genetically engineered EVs of the present invention. Other cell types include muscle cells, cardiomyocytes, smooth muscle cells, neurons, astrocytes, glial cells, B cells, T cells, dendritic cells, macrophages, neutrophils, osteoblasts, osteoclasts, adipocytes, endothelial cells, epithelial cells, cells of the kidneys, cells of the pancreas, and essentially any cell of the mammalian (for instance human) body.

In a further aspect, the present invention relates to a method of producing patient-derived EVs comprising a fusion protein comprising at least one EV polypeptide and at least one POI, the method comprising the step of administering to the cells of a patient a composition as per the present invention, whereby the cells of the patient produce the patient-derived EVs (i.e. in-vivo production of EVs rather than ex-vivo production). Suitable formulations, routes of administration, dosage amounts, regimes etc. are as described for the pharmaceutical compositions disclosed herein. The patient-derived EVs are thus genetically modified patient-derived EVs. These patient-derived EVs are heterologous to the patient, as a result of the fact that they result from the expression of the designed polynucleotide into the translated fusion protein which in turn comprises the POI (which too may be heterologous to the patient). As abovementioned, the patient cell may be any cell, preferably a liver cell such as a hepatocyte or a liver macrophage (e.g. a Kupffer cell). Various other cells and cell types in other organs than the liver may also function as the “in situ bioreactors” which are important for the method of producing the patient derived genetically engineered EVs of the present invention. Other cell types include muscle cells, cardiomyocytes, smooth muscle cells, neurons, astrocytes, glial cells, B cells, T cells, dendritic cells, macrophages, neutrophils, osteoblasts, osteoclasts, adipocytes, endothelial cells, epithelial cells, cells of the kidneys, cells of the pancreas, and essentially any cell of the patient body. Thus, in a further embodiment, the present invention relates to a patient-derived EV comprising a fusion protein comprising at least one EV polypeptide and at least one POI, wherein the patient-derived EV is manufactured by the method as described above. The present invention further relates to such genetically engineered, patient-derived EVs for use in medicine, and more specifically for use in the treatment and/or prophylaxis of diseases selected from the non-limiting group consisting of genetic diseases, hereditary diseases, lysosomal storage disorders, inborn errors of metabolism, urea cycle disorders, neuromuscular diseases, neurodegenerative diseases, cancer, infectious diseases, autoimmune diseases, cardiovascular diseases, and inflammatory diseases, and any other disease wherein these patient-derived EVs can exert a pharmacological effect.

In another aspect, the present invention relates to a method of delivering a POI to a target cell, a target organ or organ system, a target compartment, or a target tissue of a patient. The method of delivering a POI comprises the step of administering to cells (often referred to as producer cells) of a patient the compositions according to the present invention, whereby the producer cells of the patient produces patient-derived EVs comprising a fusion protein comprising the POI, wherein the patient-derived EVs deliver the POI to the target cell. Suitable formulations, routes of administration, dosage amounts, regimes etc. are as described for the pharmaceutical compositions disclosed herein. The in vivo production of genetically engineered patient-derived EVs by the producer cells result in patient-specific, i.e. autologous, engineered EVs comprising the POI, which is then delivered to cells that are the targets of the patient-derived genetically engineered EVs. The targeting of the patient-derived EVs to a given target cell may be the result of intrinsic targeting to a given cell type and/or may be a result of the introduction of a targeting moiety on the genetically engineered EV, normally via inclusion of a targeting polypeptide in the fusion protein which also comprises the POI. An example of active, engineered targeting may be the introduction of a brain targeting polypeptide into a fusion protein which is translated in a liver cell as a result of delivery of the compositions of the present invention comprising the polynucleotide to such a liver cell, followed by production of genetically engineered EVs comprising the fusion protein and the POI for exerting a pharmacological effect. The target cells into which the POI is delivered may be the same cell type as the producer cell type, or a different cell type. Target cells of the present invention include but are not limited to a cell of the liver, a central nervous system cell including a brain cell, an immune cell, a tumour cell, a muscle cell, a kidney cell, a cell of the pancreas, a cell of the heart, a lung cell, a bone marrow-derived cell, or any other cell type. Similarly, the producer cells for production of the genetically engineered, patient-derived EVs comprising the fusion protein comprising the POI can be essentially any cell in the body of a mammal, for instance a cell of the liver, a central nervous system cell including a brain cell, an immune cell, a tumour cell, a muscle cell, a kidney cell, a cell of the heart, a cell of the pancreas a lung cell, a bone marrow-derived cell, or any other cell type. As abovementioned, a preferred embodiment of the present invention relates to compositions which comprise a (i) polynucleotide cargo selected from mRNA, a circular mRNA, a linear DNA, a circular DNA, a doggybone DNA (dbDNA), a DNA plasmid, linear RNA, circular RNA, self-amplifying RNA or DNA, a viral genome, or a modified version of any of the above, and a (ii) non-viral delivery vector which is a lipid-based vector, and these non-viral delivery composition are preferred also in the context of the method for delivery POI into a target cell via secretion of EVs from a producer cell. As abovementioned, in some embodiments of the present invention, a non-viral delivery vector does not need to be selected from e.g. a lipid-based vector, a polymer-based vector, a peptide-based vector or any other active form of vector but said non-viral vector can be selected from any pharmaceutically acceptable carrier, e.g. saline solution or similar, as long as the delivery of the polynucleotide cargo into a target cell results in the expression of the polynucleotide construct into an engineered, autologous patient-derived EV which carries the fusion protein coded for by the polynucleotide.

In some preferred embodiments, more than one polynucleotide is comprised in the compositions as per the present invention, for instance more than one mRNA (i.e. two or more mRNA coding for different proteins) or one mRNA, self-amplifying RNA and one pDNA polynucleotide. Non-viral delivery vectors, including delivery vectors which are based on merely a physiologically and pharmaceutically acceptable carrier, are the preferred delivery vectors for such combinatorial polynucleotide therapies. The ability to produce in vivo genetically engineered EVs comprising a fusion protein comprising a POI binding to an mRNA in turn coding for a drug cargo (for instance an enzyme or a transmembrane protein or the like) is a highly sophisticated approach to EV-mediated delivery of said mRNA drug cargo to tissue of interest, via an autologous, long-lasting and safe approach with comparatively low cost of goods.

In another aspect, the present invention relates to a method of treatment and/or prophylaxis of a disease, disorder or condition in a subject in need thereof, wherein said method comprises administering to a subject the compositions herein, wherein translation of the polynucleotide cargo into the corresponding fusion protein results in the production of at least one EV comprising the fusion protein comprising a POI. Any disease, disorder or condition is contemplated as a suitable target for the treatment and/or prophylaxis. Treatment can include the amelioration of the disease, disorder or condition and/or an improvement in symptoms. Prophylaxis can include partial or full prevention of the disease, disorder or condition. Any adult or child human patient population may be considered for treatment or prophylaxis by the present method via any route of administration or dosage regime as defined above. Suitable formulations, routes of administration, dosage amounts, regimes etc. are as described for the pharmaceutical compositions disclosed herein.

In a further aspect, the present invention relates to a method of treating a genetic disease, disorder or condition resulting from a defect gene. Gene defects can take many forms, including mutations, deletions, truncations, duplications, chromosomal damage, deletion or duplication, and gene defects may be monogenic or polygenic. Monogenic genetic defects are particularly suitable for treatment with the patient-derived genetically engineered POI-carrying EVs of the present invention. The method for treating a disease resulting from a gene defect comprises administering to a subject a composition as per the present invention, wherein expression/translation of the polynucleotide cargo into the corresponding fusion protein results in the production of at least one extracellular vesicle (EV) comprising a POI, wherein the POI is a protein corresponding to the defective gene of the subject. Suitable formulations, routes of administration, dosage amounts, regimes etc. are as described for the pharmaceutical compositions disclosed herein. As a non-limiting example, a composition of the present invention may comprise a polynucleotide coding for an EV polypeptide fused to a POI in the form of the enzyme PAH via a self-cleaving intein. The administration of this composition to a patient suffering from the disease phenylketonuria (PKU) would result in the translation of the PAH-containing fusion protein and production of genetically engineered patient-derived EVs in which the PAH enzyme is loaded. The patient-derived EVs could for instance be produced by liver cells such as hepatocytes of the patient, which would result in secretion of liver-derived PAH-containing EVs which would then deliver the active PAH enzyme into other liver cells and also into other cells of the body. Inteins are self-cleaving polypeptides which when inserted between an EV polypeptide and a POI (like the PAH enzyme, for instance) causes release of the POI, ensuring that the enzyme is present in the EV and/or in the target cell in free, soluble form.

In embodiments of the present invention, the POI may thus be an intracellular or lysosomal enzyme or any other form of protein, for instance a membrane-associated protein or a transmembrane protein. As abovementioned, the POIs are advantageously linked to the EV polypeptide via a self-cleaving polypeptide, for instance an intein or other cis-cleaving polypeptides. The methods of the present invention are highly suitable for the treatment of various genetic diseases, with monogenic diseases being particularly suitable for treatment based on the methods of the present invention. Genetic disease, disorder or condition may be selected from among the inborn error of metabolism, the urea cycle disorders, the lysosomal storage disorders, the neuromuscular diseases, or the neurodegenerative diseases, but may in essence be any genetic disease whether monogenic or polygenic. Importantly, the present invention allows for a novel approach to treatment of these disease, through the creation of an engineered (i.e. modified), patient-derived EV product that comprises a POI via the inventive engineering of EV-producing cells to secrete systemically bioavailable patient-derived EVs as natural drug delivery vehicles.

In another aspect, the present invention relates to genetically engineered patient-derived EVs, wherein said EVs comprise a fusion protein comprising an EV polypeptide and a POI. As abovementioned, not only does patient-derived, in vivo-produced genetically engineered EVs have a distinct biodistribution profile as compared to ex vivo-manufactured genetically engineered EVs, but the COGs for manufacturing is significantly lower as these patient-derived genetically engineered EVs comprising the POI are essentially manufactured in the body of the patient, by virtue of the delivery of a composition comprising a polynucleotide encoding for the fusion protein comprising the POI which in turn results in engineered EVs comprising the fusion protein and the POI being secreted locally and/or systemically in the body of the patient. In preferred embodiments of the present invention, the genetically engineered patient-derived EVs comprise a POI that corresponds to a protein that is encoded for by a mutated, deleted, downregulated, or otherwise defect gene of the patient. This means that the engineered patient-derived EVs essentially form an autologous protein replacement therapy based on engineered EVs delivering the missing/defective protein to the target tissue. Generally, the POI may be essentially any protein of interest, for instance selected from the non-limiting group consisting of enzymes, transporters, chaperones, transmembrane proteins, structural proteins, nucleic acid-binding proteins, nucleases, recombinases, and protein-binding proteins, and any other protein that can mediate a pharmacological effect in a given disease context. The fusion proteins of the present invention are typically heterologous to the patient, i.e. they are not naturally encoded for by the patient's genome. This the result of the genetic engineering strategies that are applied to create the polynucleotide cargo which codes for the fusion protein comprising the POI. As an example, a fusion protein between a tetraspanin EV polypeptide (tetraspanins are a preferred class of EV polypeptides for the purposes of the present invention) and a given enzyme (for instance the enzyme PAH or a urea cycle enzyme) are heterologous to essentially all mammals, including all humans, as said fusion protein is not existing naturally in any mammalian system. Furthermore, the POI may itself be heterologous to the patient, which is can be the case in diseases where the POI is intended to function as a protein replacement therapy in a genetic disease context. A patient suffering from a genetic disease that results from a mutation or a deletion or similar may not have the wild-type protein present at all in the cells of the body, meaning that in these cases in addition to the fusion protein being heterologous to the patient the POI is also heterologous to the patient.

Highly surprisingly, the inventors have discovered that the genetically engineered patient-derived EVs per the present invention have a considerably longer half-life in the circulation as compared to ex vivo-produced genetically engineered EVs (even as compared to ex vivo-produced patient-derived genetically engineered EVs). This surprising technical effect is likely a function of the fact that the EVs are patient-specific (autologous) in combination with them being produced in vivo (also called in situ) in the body of the patient, which is surmised to result in a patient-specific corona associating with the genetically engineered EVs as soon as they enter the systemic circulation, for instance via the blood. Highly surprisingly, the plasma half-life in the subject of a population of the genetically engineered subject-derived EVs is normally more than 12 hours, which is at least 10 times as long as the half-life of the corresponding in vitro-manufactured EV, preferably 50 times as long, preferably 100 times as long, preferably 200 times as long, even more preferably 300 times as long, and even further preferably 500 times as long. Thus, in embodiments of the present invention the genetically engineered patient-derived EVs have a plasma half-life of more than 2 hours, preferably more than 6 hours, and even more preferably more than 24 hours, and even more preferably more than 48 hours. The plasma half-life of the patient-derived in-situ EVs may be at least about 12, 18, 24, 48, 36, 72, 100, 150, 200, 250, 300 or more hours, for instance about 5-10 hrs, about 10-15 hrs, about 5-20 hrs, about 24-48 hrs, about 24-72 hrs, about 12-72 hrs, about 12-100 hrs, about 12-200 hrs, or about 12-300 hrs. The plasma half-life of the genetically engineered patient-derived EVs can easily be measured by assaying plasma (e.g. through a blood draw) for the presence of the fusion protein and/or for the presence of the POI. Reporter proteins, such as eGFP and luciferase, can be used as POIs to facilitate the assaying and the determination of circulation time and the half-life of the genetically engineered patient-derived EVs.

As abovementioned, these in situ-produced patient-derived EVs are genetically engineered to comprise the fusion protein comprising the POI by expressing/translating in a cell of the patient a polynucleotide coding for the fusion protein comprising both the POI and an EV polypeptide, which leads to the production by the cell of EVs comprising the POI. The EV polypeptides may be selected from essentially any polypeptide that can be used to “load”, i.e. transport, the POI into an EV forming in the EV-producing cell into which the composition of the present invention is delivered. Non-limiting examples of EV polypeptides include CD9, CD53, CD63, CD81, CD54, CD50, FLOT1, FLOT2, CD49d, CD71, CD133, CD138, CD235a, AAAT, AT1B3, AT2B4, ALIX, Annexin, BASI, BASP1, BSG, Syntenin-1, Syntenin-2, Lamp2, Lamp2a, Lamp2b, TSN1, TSN3, TSN4, TSN5 TSN6, TSN7, TSPAN8, TSN31, TSN10, TSN11, TSN12, TSN13, TSN14, TSN15, TSN16, TSN17, TSN18, TSN19, TSN2, TSN4, TSN9, TSN32, TSN33, TN FR, TfR1, syndecan-1, syndecan-2, syndecan-3, syndecan-4, CD37, CD82, CD151, CD224, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, CLIC1, CLIC4, interleukin receptors, CD2, CD3 epsilon, CD3 zeta, CD13, CD18, CD19, CD30, CD34, CD36, CD40, CD40L, CD44, CD45, CD45RA, CD47, CD53, CD86, CD110, CD111, CD115, CD117, CD125, CD135, CD184, CD200, CD279, CD273, CD 274, CD362, COL6A1, AGRN, EGFR, FPRP, GAPDH, GLUR2, GLUR3, GP130, GPI anchor proteins, GTR1, HLAA, HLA-DM, HSPG2, ITA3, Lactadherin, L1CAM, LAMB1, LAMC1, LIMP2, MYOF, ARRDC1, ATP2B2, ATP2B3, ATP2B4, BSG, IGSF2, IGSF3, IGSF8, ITGB1, ITGA4, ATP1A2, ATP1A3, ATP1A4, ITGA4, SLC3A2, ATP transporters, ATP1A1, ATP1B3, ATP2B1, LFA-1, LGALS3BP, Mac-1 alpha, Mac-1 beta, MFGE8, a member of the myristoylated alanine rich Protein Kinase C substrate (MARCKS) protein family such as MARCKSL1, matrix metalloproteinase-14 (MMP14), PTGFRN, BASP1, MARCKS, MARCKSL1, PTGFRN, PRPH2, ROM1, SLIT2, SLC3A2, SSEA4, STX3, TCRA, TCRB, TCRD, TCRG, TFR1, UPK1A, UPK1B, VTI1A, VTI1B, and any other EV polypeptide, and any combinations, derivatives, domains, variants, mutants, or regions thereof

The present invention does not only allow for significant modularity and optionality as it relates to the EV polypeptides, but numerous types of proteins can be used as the POI in the context of the present invention. Non-limiting examples of suitable POIs include enzymes, transporters, chaperones, transmembrane proteins, structural proteins, nucleic acid-binding proteins, nucleases, recombinases, and protein-binding proteins, and essentially any other protein that can mediate a pharmacological effect itself, or that can bind to another agent which in turn mediates a pharmacological effect. For instance, the POI may be an RNA-binding protein which binds to a given RNA cargo molecule (such as an mRNA or an shRNA or a miRNA) and transports the RNA cargo into the genetically engineered patient-derived EV. The ability to incorporate additional pharmacologically active cargo biomolecules into the in situ produced genetically engineered EVs by using the POI allows for a plethora of applications. In one embodiment, more than one polynucleotide cargo can be incorporated into the compositions here, in order to allow for EV loading of via a first polynucleotide the fusion protein comprising the POI, via a second polynucleotide another protein which is bound by the POI and thus is loaded into the EV produced by the EV-producing cell in vivo, and via a third and further polynucleotides additional cargo molecules may be loaded into the EVs. In one advantageous embodiment, the polynucleotide cargo comprised in the compositions of the present invention codes for both a fusion protein comprising a POI (which is then an RNA-binding protein) and for an RNA molecule such as an mRNA, an shRNA, or a miRNA. The POI being an RNA-binding protein means that said RNA-binding protein can be designed so as to bind a specific sequence on a given RNA molecule, for instance in the UTRs of an mRNA. This modular engineering approach means that the in situ produced patient-derived engineered EV utilises its POI to bind to a given mRNA cargo, allowing for EV-mediated transport of the mRNA into target tissues in the body. As in the example above, the POI and the RNA drug molecule (i.e. the mRNA, the self-amplifying RNA, the shRNA, or the miRNA) can be encoded for by a single polynucleotide (for instance a DNA plasmid or any other form of polynucleotide which is capable of coding for both a protein in the form of a POI and an RNA molecule, for instance in the form of an mRNA. Furthermore, bicistronic and other forms of multicistronic polynucleotides may be utilised to code for more than one POI, for instance to code for a POI which binds to another protein which in turn forms the drug in question.

In addition to the EV polypeptide and the POI, as abovementioned additional polypeptide domains may advantageously by included in the fusion protein, to for instance (i) targeting polypeptides to mediate cell type-specific targeting, (ii) serum albumin-binding domains to allow for even further extended plasma half-life by binding to serum albumin, (iii) release polypeptides such as cis-cleaving polypeptides (e.g. inteins) so as to release the POI from the fusion protein, (iv) nucleic acid-binding domains for to binding of various forms of nucleic acid-based molecules, etc. As abovementioned, essentially any cell type can produce the genetically engineered patient-derived EVs as a result of translation of the polynucleotide of the compositions described herein, however certain cells are particularly productive in terms of EV yield. The liver is a metabolically active organ which secretes considerable amounts of EVs and can be used as an efficient “in situ” (interchangeably termed “in vivo”) bioreactor for production of engineered patient-derived EVs comprising the fusion protein comprising the POI. Liver cells of particular utility for the present invention include hepatocytes and liver macrophages.

The genetically engineered patient-derived EVs per the present invention have significant utility for use in medicine. More in detail, the genetically engineered patient-derived EVs can be for use in the treatment of diseases selected from the non-limiting group consisting of genetic diseases, lysosomal storage disorders, inborn errors of metabolism, urea cycle disorders, neuromuscular diseases, neurodegenerative diseases, cancer, infectious diseases, autoimmune diseases, kidney diseases, liver diseases, cardiovascular diseases, and inflammatory diseases, as well as any other disease wherein a suitable POI can exert a pharmacological effect. Non-limiting examples of diseases in which the patient-derived EVs of the present invention could advantageously be applied include the following examples: Crohn's disease, diabetes mellitus type 1, Graves' disease, inflammatory bowel disease, multiple sclerosis, psoriasis, rheumatoid arthritis, systemic lupus erythematosus) ulcerative colitis, ankylosing spondylitis, sarcoidosis, idiopathic pulmonary fibrosis, psoriasis, tumour necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy, Becker's muscular dystrophy and other muscular dystrophies, inborn errors of metabolism including disorders of carbohydrate metabolism e.g., G6PD deficiency galactosemia, hereditary fructose intolerance, fructose 1,6-diphosphatase deficiency and the glycogen storage diseases, disorders of organic acid metabolism (organic acidurias) such as alkaptonuria, 2-hydroxyglutaric acidurias, methylmalonic or propionic acidemia, multiple carboxylase deficiency, disorders of amino acid metabolism such as phenylketonuria (PKU), maple syrup urine disease, glutaric acidemia type 1, aminoacidopathies e.g., hereditary tyrosinemia, nonketotic hyperglycinemia, and homocystinuria, Hereditary tyrosinemia, Fanconi syndrome, Primary Lactic Acidoses e.g., pyruvate dehydrogenase, pyruvate carboxylase and cytochrome oxidase deficiencies, disorders of fatty acid oxidation and mitochondrial metabolism such as short, medium, and long-chain acyl-CoA dehydrogenase deficiencies also known as Beta-oxidation defects, Reye's syndrome, Medium-chain acyl-coenzyme A dehydrogenase deficiency (MCADD), MELAS, MERFF, pyruvate dehydrogenase deficiency, disorders of porphyrin metabolism such as acute intermittent porphyria, disorders of purine or pyrimidine metabolism such as Lesch-Nyhan syndrome, disorders of steroid metabolism such as lipoid congenital adrenal hyperplasia, congenital adrenal hyperplasia, disorders of mitochondrial function such as Kearns-Sayre syndrome, disorders of peroxisomal function such as Zellweger syndrome and neonatal adrenoleukodystrophy, congenital adrenal hyperplasia or Smith Lemli-Opitz, Menkes syndrome, neonatal hemochromatosis, urea cycle disorders such as N-acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), all of the lysosomal storage diseases, for instance Alpha-mannosidosis, Betamannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Mucopolysaccharidosis, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, etc. cystic fibrosis, primary ciliary dyskinesia, pulmonary alveolar proteinosis, ARC syndrome, Ret syndrome, neurodegenerative diseases including Alzheimer's disease, Parkinson's disease, GBA associated Parkinson's disease, Huntington's disease and other trinucleotide repeat-related diseases, prion diseases, dementia including frontotemporal lobe dementia, ALS, motor neuron disease, multiple sclerosis, cancer-induced cachexia, anorexia, diabetes mellitus type 2, Limb Girdle type 2A, Limb Girdle Type 2D, spinal muscular atrophy respiratory distress type I (SMARD1), Spinal bulbar muscular atrophy (SBMA), and various cancers. Virtually all types of cancer are relevant disease targets for the present invention, for instance, acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumour, Brainstem glioma, Brain cancer, Brain tumour (cerebellar astrocytoma, cerebral astrocytoma/malignant glioma, ependymoma, medulloblastoma, supratentorial primitive neuroectodermal tumours, visual pathway and hypothalamic glioma), Breast cancer, Bronchial adenomas/carcinoids, Burkitt's lymphoma, Carcinoid tumour (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumour, Endometrial cancer, Ependymoma, Esophageal cancer, Extracranial germ cell tumour, Extragonadal Germ cell tumour, Extrahepatic bile duct cancer, Eye Cancer (Intraocular melanoma, Retinoblastoma), Gallbladder cancer, Gastric (Stomach) cancer, Gastrointestinal Carcinoid Tumour, Gastrointestinal stromal tumour (GIST), Germ cell tumour (extracranial, extragonadal, or ovarian), Gestational trophoblastic tumour, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumour), Ovarian germ cell tumour, Ovarian low malignant potential tumour, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumours, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumours sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumour, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumour.

In another aspect of the present invention, as abovementioned, the patient-derived genetically engineered EVs may be manufactured by a method comprising administering to a subject (e.g. a patient) a composition, wherein expression/translation of the polynucleotide cargo (which is comprised in the composition) into the corresponding fusion protein results in the production of population of patient-derived genetically engineered EVs comprising the fusion protein which in turn comprises the POI. Importantly, the ability to design and manufacture a composition for polynucleotide delivery using viral or non-viral nanoparticle delivery methods enable harnessing the modularity and versatility of engineered EVs, while reducing COGs, extended circulatory half-life of the engineered EVs, and minimize or completely abrogate the risk of innate or adaptive immunogenicity or any other safety concerns associated with in vitro-manufactured EV therapeutics.

Those skilled in the art will recognize or will be able to ascertain using no more than routine experimentation equivalents to the specific embodiments in accordance with the present invention as described herein. The scope of the present invention is not intended to be limited by the description above and not by the following examples and experiments. The invention and its various aspects, embodiments, alternatives, options, and variants will now be further exemplified and illustrated with the enclosed non-limiting examples, which may be modified considerably using no more than route experimentation without departing from the scope and the gist of the invention.

### Example 1: Genetically Engineered Therapeutic EVs Produced In Situ Provide Long Term Pharmacological/Therapeutic Effect in a Mouse Model of Colitis

In order to test the therapeutic effect of EVs engineered to comprise a TNFR decoy as the POI comprised in a fusion protein with an EV polypeptide (in this experiment various tetraspanins (CD9, CD63 and CD81) were tested with positive results, with data shown for CD63) when those EVs are produced in vivo (in situ), a mouse model of colitis was used. TNBS-induced colitis is a well-understood Balb/C mouse model which simulates the cytokine storm, the diarrhea, weight decrease, and gut inflammation seen in IBD patients. The EVs produced were designed and genetically engineered to express a signalling incompetent TNF receptor which would attenuate the inflammation in the colitis model.

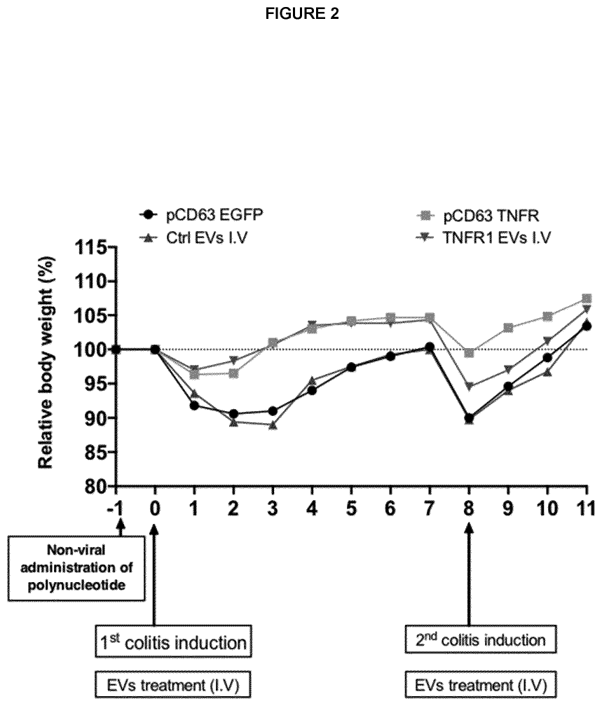

FIG. 2 shows a comparison of two different methods of delivering TNFR decoys. Firstly, by non-viral administration of a plasmid-containing drug delivery composition to transform the liver cells (surmised to be a combination of both liver macrophages and hepatocytes) of mice and secondly by intravenous administration of EVs engineered to comprise TNFR decoys. As abovementioned, the fusion protein expressed from the plasmid DNA polynucleotide cargo was based on fusing an EV polypeptide in the form of a tetraspanin with the TNF receptor as the POI.

The treatment groups were as follows:
a) Non-viral delivery of plasmid DNA as the polynucleotide cargo, with the pDNA comprising the signalling incompetent TNF receptor (TNF decoy as the POI) fused to an EV polypeptide (data shown for CD63)
b) IV injection of in vitro-produced EVs comprising the same TNFR decoy fusion protein
c) Non-viral delivery of plasmid DNA as the polynucleotide cargo, with the pDNA construct not coding for the POI but merely the EV polypeptide (data shown for CD63) (as a negative control)
d) IV injection of in vitro-produced EVs lacking the TNFR protein of interest in the fusion protein (as a negative control)

FIG. 2 shows that delivery of the TNFR decoy by non-viral administration of a composition comprising a pDNA polynucleotide resulted in production of autologous, genetically engineered EVs carrying the fusion protein with the POI, with the POI specifically conferring long term protection against TNBS-induced colitis and outperforming engineered EVs that were manufactured ex vivo and administration systemically via IV administration. It is important to note that engineered EV treatment requires repeat dosing and still does not achieve the same pharmacological effect as autologous engineered EVs produced in situ, whereas, a single non-viral administration of a composition comprising the TNFR-CD63 plasmid as the polynucleotide cargo is still therapeutically effective 9 days after initial and challenge treatment and provides improved therapeutic effect compared to ex vivo-manufactured EVs delivered simultaneously with the second induction of colitis.

### Example 2: Biodistribution Experiment Showing that Engineered EVs Produced In Situ in the Liver are Detectable in Wide Range of Organs and Plasma

NMRI mice were injected with human CD63-NanoLuc pDNA or NanoLuc only pDNA by hydrodynamic infusion (HI) as a proof-of-concept of liver cell-derived engineered EV production. The HI administration delivers the plasmid DNA polynucleotide to the liver and specifically to hepatocytes, resulting in the production of the fusion protein CD63-NanoLuc or NanoLuc alone in the liver cells. The mice transformed with the CD63-NanoLuc pDNA polynucleotide then produce autologous engineered EVs that comprise the humanCD63-Nanoluc fusion protein by virtue of the EV polypeptide (which transports the fusion protein into the autologously generated EVs). The livers of mice transformed with the NanoLuc only plasmid polynucleotide cargo will express only NanoLuc, which is actively loaded into the EVs produced by those liver cells.

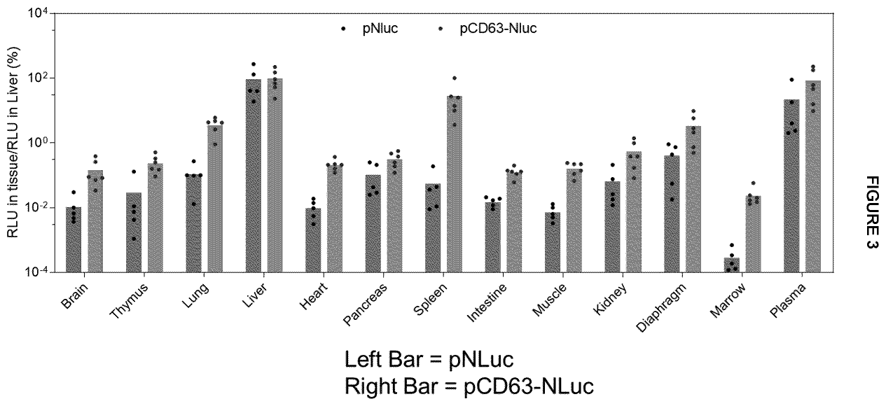

Organs/plasma were collected after 48 hours and analysed to determine the relative luminescence units (RLUs) of NanoLuc present in different organs. FIG. 3 shows RLUs in tissue/RLU in liver and shows a clear distribution shift from liver to tissues such as brain, muscle and plasma supportive of in situ engineered EV production and demonstrating that EVs comprising the fusion protein are produced by liver cells, released and then taken up by other organs.

The presence of human CD63-NanoLuc EVs in plasma confirms in situ production and release as well as indicates that the EVs generated persisted in the plasma for at least 48 hrs, meaning that their half-life is significantly longer than the plasma half-life of ex vivo manufactured EVs. Similar data was obtained with LNP (specifically DLin-MC3-DMA)-mediated delivery of a modified mRNA polynucleotide (with pseudouridine (ψ) and 5-methylcytidine (m5C) modifications) coding for the humanCD63-NanoLuc fusion protein and a similar biodistribution profile was observed, with extended half-life of the liver cell-derived engineered EVs as compared to in vitro-manufactured engineered EVs.

### Example 3: Comparison of the Level of Enzymatic Activity in Mouse Plasma Over Time of (i) Mouse In Situ Produced EVs Comprising Human CD63-NanoLuc and (ii) the Corresponding In Vitro-Manufactured EVs

In order to investigate the half-life of IV injected engineered EVs vs in situ autologously produced engineered EVs, NMRI mice were either injected IV or produced in situ via non-viral delivery methods of compositions comprising a vector and a polynucleotide cargo (in this case LNP mediated delivery of mRNA and HI of pDNA) coding for the EV fusion protein. Plasma was collected at various time points for flow cytometry analysis to determine the amount of engineered EVs in circulation. The presence of the engineered EVs in plasma was assayed by flow cytometry using anti-human pan-tetraspanin antibodies labelled with APC.

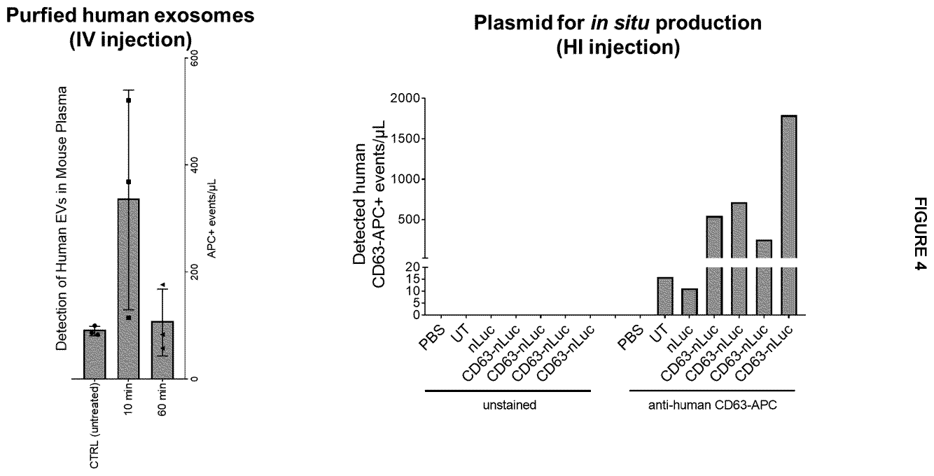

In a first experiment 10\hat{ }11 unlabelled HEK293 EVs were injected IV into mice which were then sacrificed at 10 and 60 minutes and plasma collected and analysed by flow cytometry. FIG. 4 shows that IV injected unlabelled human EVs can be detected in mouse plasma after 10 minutes and that the levels are well above background. However, by the 60 min timepoint the EVs are rapidly taken up by cells and are already near the lower level of detection in plasma.

In a second experiment non-viral administration of human CD63-NanoLuc polynucleotides versus polynucleotides with NanoLuc alone was compared. FIG. 4 also shows that in situ produced autologous genetically engineered humanCD63-NanoLuc EVs were abundant in plasma even after 48 hrs. When comparing the two experiments it can be seen that the APC+events/uL in the IV injection of ex vivo-manufactured EVs vs the in situ produced EVs show that levels of EVs in the in vivo-engineered mice after 48 hrs were higher than at 10 min after injection of ex vivo-manufactured EVs. This effect was observed over 4 repeats using various delivery methods.

### Example 4: Effect of Albumin-Binding Polypeptides on Half-Life of In Situ Produced Engineered EVs

It is known that the presence of albumin (usually in the form of a fusion protein) can increase the circulation time of injected biologic drugs. One of the present inventors have also previously invented and patented an approach whereby engineering onto the surface of EVs albumin-binding polypeptides (often called albumin-binding domain (ABD)) can cause albumin to attach to the outer surface of the EVs and thus increase its half-life. It was predicted that extended half-life and altered biodistribution of ABD-engineered EVs would also be seen when the EVs were produced in situ following non-viral or viral delivery of a composition comprising a polynucleotide coding for an EV polypeptide, a POI and an ABD domain placed on the outside of the subsequently autologously produced genetically engineered EVs. To test this strategy non-viral methods of delivery of a composition comprising pDNA (HI and LNP mediated delivery) coding for human CD63-ABD-NanoLuc, CD63-NanoLuc or NanoLuc plasmid alone were tested in mice. 72 hours after administration of the compositions NanoLuc levels were assayed and analyzed.

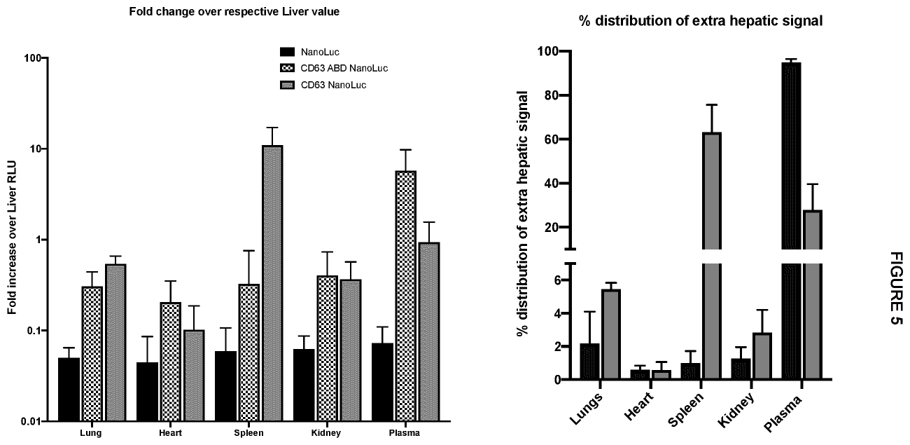

FIG. 5 shows that in situ production of autologous genetically engineered EVs comprising the CD63-ABD fusion protein led to improved tissue distribution with both CD63 constructs but that the presence of the ABD domain in the construct led to less uptake in spleen and more importantly considerably higher EV levels in plasma, meaning the presence of ABD in the construct increases the plasma half-life of the in situ produced engineered EVs. Almost 100% of the ABD EVs detected at 72 hrs were still in circulation. This indicates that ABD is beneficial for altering both the biodistribution of EVs by crucially increasing the plasma concentration and thereby significantly increasing the circulation time of in situ produced EVs. This discovery is especially useful in the treatment of extrahepatic diseases, for instance disease and conditions that require the engineered EVs to deliver a given drug cargo (e.g. a POI or any other agent to which the POI can bind and which can exert a pharmacological effect) across a tissue barrier, such as the blood brain barrier. However, increased circulation time is important for targeted delivery to any organ and the ability to reduce liver clearance by incorporating an ABD combined with engineered EV production in situ represents a transformative approach.

### Example 5: Delivery of Various Polynucleotide Acid Cargos by Additional Delivery Methods

Following the discovery that plasmid delivered by hydrodynamic injection and mRNA/pDNA delivery by LNP was capable of transforming liver cells in vivo to produce therapeutic engineered EVs with strong and long lasting therapeutic effects it was postulated that other delivery mechanisms such as viral vector delivery (such as AAVs or lentivirus) or other lipid-based delivery vectors (such as other LNPs, lipidoids, or liposomes)) or protein/peptide-based delivery vehicles (such as CPPs) to form a polyplex with the cargo could also be used to deliver plasmids or any other type of polynucleotide cargoes (including mRNA, self-replicating RNA, naked AAV genome, etc.). It is also thought that delivery of a wide range of different polynucleotide cargoes is possible not just to liver but to other organs such that in situ production is possible from organs such as the brain or the central nervous system, muscle cells, or immune system cells, among other EV-producing cell types.

a) mRNA Delivery by LNP

As abovementioned, LNP-mediated mRNA delivery has shown to efficiently produce genetically engineered EVs comprising the fusion protein coded for by the mRNA polynucleotide. In order to comprehensively optimize delivery of mRNA, numerous versions of modified mRNA may be synthesized (for instance by the mRNA supplier TriLink) and formulated into lipid nanoparticles. The following modified mRNA constructs may be synthesized, with varying degrees of modifications using for instance 5-methylcytidine (m5C), 5-methyluridine (m5U), 2-thiouridine (s2U), 5-methoxyuridine (5moU), pseudouridine (ψ), and/or N1-methylpseudouridine (m1ψ):
NanoLuc mRNA
Human CD63-Nanoluc mRNA
Human CD63-Nanoluc-ABD mRNA
Mouse CD63-Nanoluc mRNA
Mouse CD63-Nanoluc-ABD mRNA

These mRNA constructs can then be formulated into lipid-based non-viral delivery vectors (e.g. DlinDMA, Dlin-MC3-DMA, C12-200, cKKE12, 5A2-SC8, or 7C1) and tested in mice and non-human primates. It is hypothesized that an optimized composition combining modified mRNA with a non-viral lipid-based delivery vectors will be able to result in enhanced delivery and translation of the polynucleotide cargo coding for the fusion protein comprising the EV polypeptide and the POI fusion protein and the resultant secretion of the autologous engineered EVs will be even higher and more sustained over time (i.e. even more favourable PK/PD profile than seen in e.g. Example 2-4).

b) mRNA Delivery by CPP to CNS

In order to test the efficacy of localized delivery of mRNA delivered by cell penetrating peptides to the CNS, modified mRNA may be synthesized (for instance by the mRNA supplier TriLink) and formulated together with the CPP delivery vehicle (including Pepfect peptides, TP10, transportan, penetratin, Tat, or other CPPs). The following modified mRNA constructs may be synthesized, with varying degrees of modifications using for instance 5-methylcytidine (m5C), 5-methyluridine (m5U), 2-thiouridine (s2U), 5-methoxyuridine (5moU), pseudouridine (ψ), and/or N1-methylpseudouridine (m1ψ):
NanoLuc mRNA
Human CD63-Nanoluc mRNA
Human CD63-Nanoluc-ABD mRNA

It is hypothesized that delivery of mRNA by a CPP to a specific organ, such as the central nervous system including the brain, would result in the production of long lasting engineered EVs in situ in the same way as non-viral delivery methods have been shown to deliver plasmid DNA and mRNA to the liver for sustained EV production in Examples 2-4. The advantage of localized delivery of the mRNA (or any other polynucleotide cargo according to the invention) is that a single delivery to a hard to access organ, such as the brain, will allow the mRNA or other polynucleotide to produce therapeutic engineered EVs in situ at a site/organ which may otherwise be protected or excluded from access by circulating drugs, such as via protection by blood brain barrier in the case of the brain. This means that a single injection into the organ of interest (e.g. brain) will not only allow much higher levels of the produced engineered EVs carrying the fusion protein to be taken up by target cells than would ordinarily be possible due to the protection by the BBB but ensures that there is a sustained delivery of the in situ produced EVs carrying the drug (i.e. the POI or any other drug with which the POI interacts) by the organ of interest.

c) Viral Delivery Vectors for Delivery of the EV-Encoding Polynucleotide

Single stranded DNA or RNA polynucleotides encoding for a fusion protein comprising an EV polypeptide and the POI may be incorporated into a virus, such as an adeno-associated virus (AAV) or lentivirus. This viral “EV gene therapy” approach (which can advantageously be applied with a focus on liver-directed gene therapy using e.g. lentivirus or the above referenced liver-tropic AAV serotypes or focused on the CNS by utilising CNS-tropic AAV serotypes) may then be used to treat animal models to show that in place of the non-viral administration of Examples 2-4, polynucleotide cargos can be delivered to transform liver, CNS or even muscle cells (or any other cell type in any organ that can be targeted by a suitable viral vector) and produce autologous engineered EV therapeutics carrying various types of payloads and exhibiting improved half-lives.

### Example 6: In-Vivo Biodistribution of In-Situ Produced Exosomes Following Delivery of mRNA by Lipid Nanoparticle (LNP)

Following the success of the delivery of plasmid DNA by hydrodynamic injection as detailed in the above Examples the present inventors then wished to confirm that this effect is achievable when delivering alternative nucleic acid cargos encoding the required fusion protein and additionally delivering such nucleic acids by alternative vehicles.

It was hypothesized that delivery of mRNA by lipid nanoparticles could be equally effective as plasmid DNA delivered by HI. In order to test this theory the present inventors designed an experiment whereby mRNA encoding human CD63-ABD-luc, human CD63-luc, mouse CD63-ABD-luc, mouse CD63-luc or luc alone was encapsulated in LNPs by passing the lipid mixture (Phospholipid, Ionizable lipid, cholesterol and PEG lipid) and the mRNA through a nanofluidic device. To prove the feasibility of the theory, mice were treated by intravenous administration of the LNPs encapsulating mRNA (1 mg/kg, 5 mice per group). The biodistribution of the nanoluc luciferase, which was translated from the delivered mRNA, was then analyzed after 72 hrs by ex vivo luciferase assay. Briefly, harvested #issues were lysed in 1 ml 0.1% TritonX-100 in PBS using a Qiagen Tissue Lyser II. Tissue lysate was then diluted 1:10 in 0.1% TritonX-100 and 10 μl of tissue lysate was added into white-walled 96-well plates along with 30 μL Nano-Glo substrate diluted 1:50 in the provided buffer (Nano-Glo Luciferase Assay System: Promega).

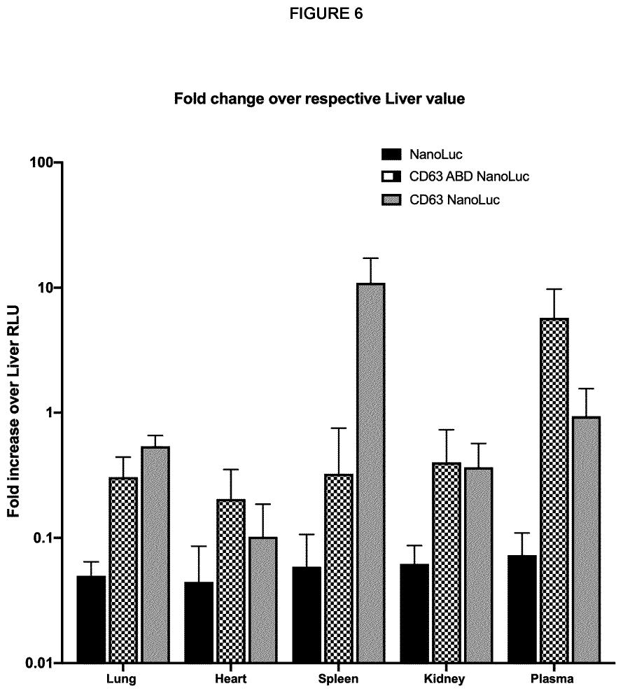

The data in FIG. 6 show the biodistribution of in-situ produced EVs following delivery of mRNA (encoding human CD63-luc, human CD63-ABD-luc or luc alone) encapsulated in a lipid nanoparticle. This figure shows production of in-situ exosomes is possible by delivery of mRNA (as well as plasmid delivery as per earlier experiments) and additionally shows that delivery of an in-situ construct whether mRNA or plasmid is possible by encapsulation in an LNP to a wide variety of different organ types.

The presence of NanoLuc in organs and plasma confirmed in situ production and release of EVs comprising the fusion protein. This also shows that the generated EVs persisted in the plasma for at least 72 hrs, meaning that their half-life is significantly longer than the plasma half-life of ex vivo manufactured EVs. Furthermore this shows that in situ production of autologous genetically engineered EVs comprising the CD63-ABD fusion protein led to improved tissue distribution with both CD63 constructs and that the presence of the ABD domain in the construct led to considerably higher EV levels in plasma, meaning the presence of ABD in the construct increased the plasma half-life of the in situ produced engineered EVs. This indicates that ABD is beneficial for altering both the biodistribution of EVs by crucially increasing the plasma concentration and thereby significantly increasing the circulation time of in situ produced EVs. Once again, this discovery is especially useful in the treatment of extrahepatic diseases, for instance disease and conditions that require the engineered EVs to deliver a given drug cargo (e.g. a POI or any other agent to which the POI can bind and which can exert a pharmacological effect) across a tissue barrier, such as the blood brain barrier.

### Example 7: Effect of Albumin-Binding Polypeptides on Half-Life of In Situ Engineered EVs Produced Following mRNA Delivery by LNP

Following from the data in FIG. 6 showing that the delivery of mRNA encoding the in-situ construct resulted in in-situ produced EVs with a long plasma half-life of 72 hrs or more the present inventors wished to test whether similar results to those seen in FIG. 5 (Example 4) could also be achieved by incorporation of an albumin binding domain (ABD) into the in-situ construct, using a time course experiment to study the kinetics in more detail.

To test whether the ABD domain would again increase the plasma half-life of the in-situ EVs mice were treated by IV administration of LNPs (1 mg/kg) encapsulating mRNA encoding human CD63-ABD-luc, human CD63-luc, mouse CD63-ABD-luc, mouse CD63-luc or nanoluc alone (N=5 per group). mRNA encapsulated in LNPs was prepared by passing the lipid mixture (Phospholipid, Ionizable lipid, cholesterol and PEG lipid) and the mRNA through a nanofluidic device. The luciferase levels of the translated constructs were then analyzed in plasma after 24 hrs, 48 hrs and 72 hrs. Briefly, harvested #issues were lysed in 1 ml 0.1% TritonX-100 in PBS using a Qiagen Tissue Lyser II. Tissue lysate was then diluted 1:10 in 0.1% TritonX-100 and 10 μl of tissue lysate was added into white-walled 96-well plates along with 30 μL Nano-Glo substrate diluted 1:50 in the provided buffer (Nano-Glo Luciferase Assay System: Promega).

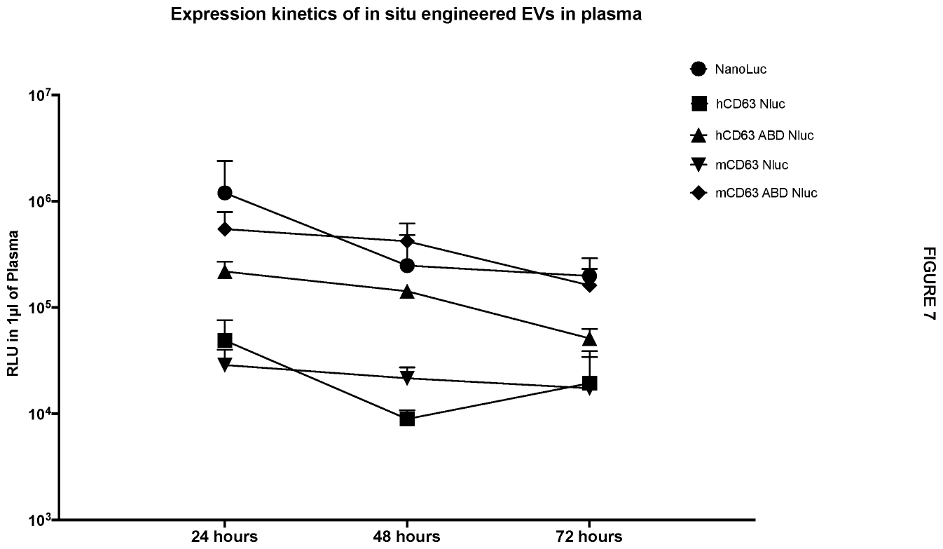

The expression kinetics results are shown in FIG. 7. FIG. 7 demonstrates that in situ production of autologous genetically engineered EVs comprising the CD63-ABD fusion protein lead to improved retention of those EVs in plasma, corroborating earlier findings. This shows that, similar to the data in FIGS. 5 and 6, the presence of ABD in the construct increased the plasma half-life of the in situ produced engineered EVs. This discovery is especially useful in the treatment of extrahepatic diseases, for instance disease and conditions that require the engineered EVs to deliver a given drug cargo (e.g. a POI or any other agent to which the POI can bind and which can exert a pharmacological effect) across a tissue barrier, such as the blood brain barrier. However, increased circulation time is important for targeted delivery to any organ and the ability to reduce liver clearance by incorporating an ABD combined with engineered EV production in situ represents a transformative approach.

### Example 8: Comparison of Plasma Pharmacokinetics of In-Situ Vs Purified EVs

In order to further investigate the plasma pharmacokinetics of in-situ EVs produced following delivery of mRNA an experiment was conducted to compare pharmacokinetics of in-situ vs purified EVs. Briefly, mice were injected with either LNPs encapsulating mRNA encoding human CD63-luc (2 mg/kg) or 1×10¹¹ human CD63-luc engineered HEK293T EVs. Animals were blood sampled at different time points and plasma was analysed by luciferase assay to determine the EV levels.

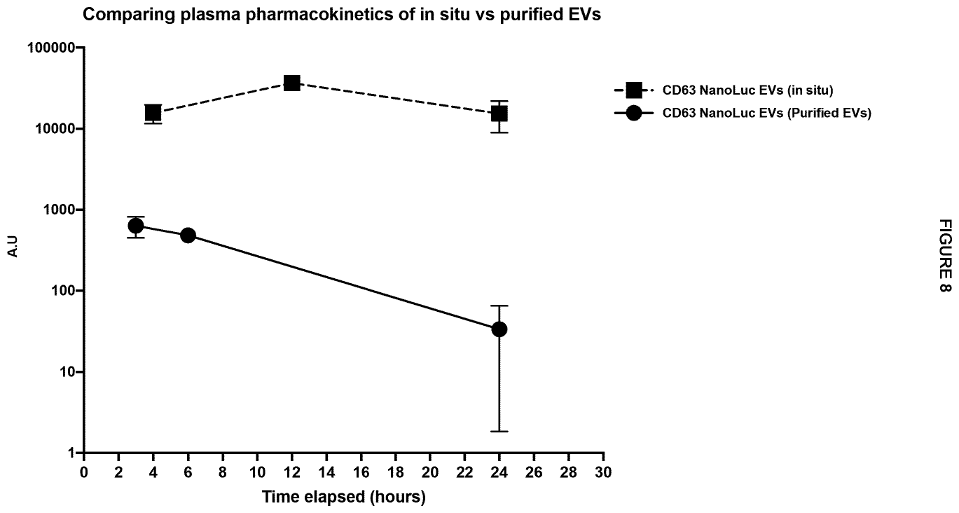

FIG. 8 shows a comparison of the plasma pharmacokinetics of EVs expressing the CD63-Nanoluc construct when the EVs were produced either a) by in-situ (in-vivo) production following delivery of an mRNA encoding the in-situ construct or b) purified EVs produced ex-vivo (produced from cell culture) and then administered by IV injection.

It can be seen by comparing the results for ex vivo-manufactured EVs vs the in situ produced EVs that the levels of EVs in the in vivo-engineered mice are consistently higher than those when ex-vivo manufactured EVs are delivered. This effect becomes more pronounced over the 24 hr time course.

### Example 9: Testing Fusion Proteins Comprising Alternative EV Polypeptides

The present inventors then tested a range of alternative EV proteins to investigate which EV proteins are the best scaffold for use in the in-situ context. The following constructs were tested:
TfR VHH IL6ST ABD FDN TFR Nluc
TfR VHH IL6ST ABD FDN NST TFR Nluc
TfR VHH IL6ST ABD LZ TFR Nluc
TfR VHH ABD PTGFRN Nluc

(TfR VHH=VHH targeting transferrin receptor, IL6ST=interleukin6 signal transducer, FDN=foldon domain, NST=N terminal syntenin, TFR=transferrin receptor, ABD=albumin binding domain, LZ=leucine zipper, PTGFRN=Prostaglandin F2 Receptor Inhibitor, Nluc=nanoluc luciferase)

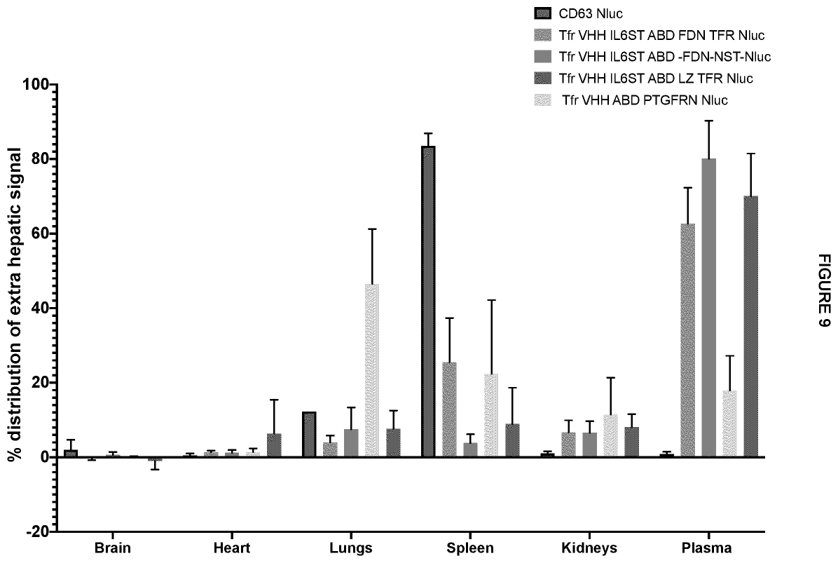

50 μg of pDNA encoding the above constructs in approximately 2 ml (8% body weight) saline was administered intravenously to mice (N=5). The biodistribution of the nanoluc luciferase incorporated into the in-situ produced EVs was then analyzed in plasma and tissue lysate at 72 hours. The data in FIG. 9 show the biodistribution of in-situ produced EVs following delivery of pDNA encoding the above constructs. Briefly, harvested tissues were lysed in 1 ml 0.1% TritonX-100 in PBS using a Qiagen Tissue Lyser II. Tissue lysate was then diluted 1:10 in 0.1% TritonX-100 and 10 μl of tissue lysate was added into white-walled 96-well plates along with 30 μL Nano-Glo substrate diluted 1:50 in the provided buffer (Nano-Glo Luciferase Assay System: Promega).

The presence of NanoLuc in organs and plasma confirms in situ production and release of EVs comprising the different fusion proteins. This also shows that the generated EVs persisted in the plasma for at least 72 hrs. Importantly, this figure shows production of in-situ exosomes is achieved by a range of different constructs using a number of different EV proteins showing that this phenomenon is shared by a wide range of different EV proteins.

### Example 10: In-Situ EV Delivery of Therapeutic Protein for Treatment of Colitis

Following the success of the therapeutic delivery by in-situ EVs shown in Example 1, the present inventors wished to test the ability of alternative in-situ produced EVs to deliver therapeutic proteins to treat disease.

The TNBS-induced mouse model of colitis (Scheiffele et al., Curr Protoc Immunol. 2002, Chapter 15: Unit 15.19) was used. This model is a well-understood Balb/C mouse model which simulates the cytokine storm, the diarrhoea, weight decrease, and gut inflammation seen in IBD patients.

VSVG-intein-Super-repressor ikBa (VSVG=Vesicular stomatitis virus G, ikBa=nuclear factor of kappa light polypeptide gene enhancer in B-cells inhibitor, alpha) is known to modulate NFkB and relieve inflammation. Constructs comprising CD63 or VSVG as the EV protein fused to super-repressor ikBa (the therapeutic POI) were generated with a self-cleaving intein inbetween so that the POI was releasable in soluble form.

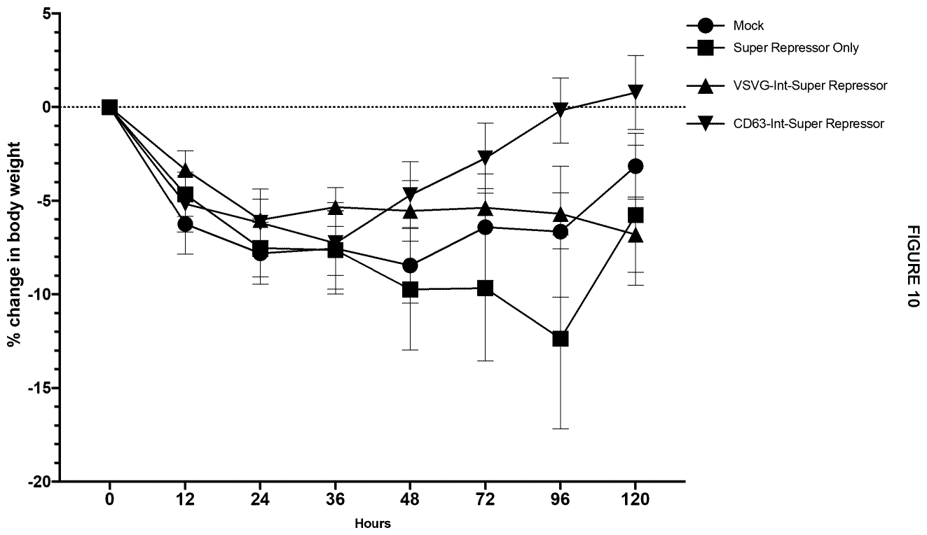

50 μg of pDNA encoding the constructs (CD63-intein-Super-repressor and VSVG-intein-Super-repressor) in approximately 2 ml (8% body weight) saline were injected IV into mice, N=9. 24 hours later animals were induced with TNBS colitis. Animals were weighed and scored everyday for the next 5 days. The results are provided in FIG. 10 and show that delivery of the super-repressor by non-viral administration of a composition comprising a pDNA polynucleotide resulted in production of autologous, genetically engineered EVs carrying the fusion protein with the super-repressor, and that the super-repressor had a therapeutic effect, conferring long term protection against TNBS-induced colitis vs control treatment.

### Example 11: In-Situ Produced EVs Reduce Inflammatory Cytokine Levels in Colitis Model

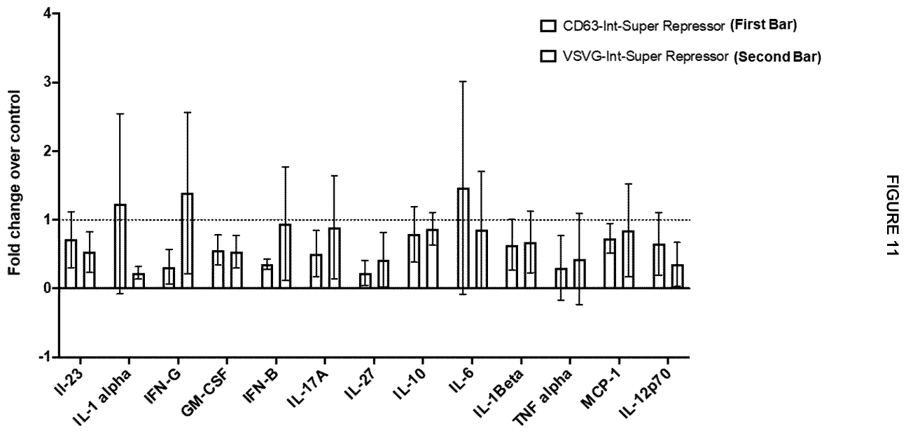

Following the experiment described in Example 10 the present inventors then studied the levels of pro-inflammatory cytokines in mice using the same colitis model treated with the same constructs as Example 10 (CD63-intein-Super-repressor and VSVG-intein-Super-repressor). BALB/c mice were induced with TNBS colitis on day 0 (N=9) by intrarectal administration of 30 μl TNBS+42.1 μl 95% ethanol+27.9 μl H₂O per mouse. Plasma levels of 13 different proinflammatory cytokines (IL-23, IL-1alpha, INF-G, GM-CSF, INF-B, IL17A, IL27, IL-10, IL-6, IL-1Beta, TNF-alpha, MCP-1 and IL-12p70) were measured on day 5 p.d.i. It can be seen from FIG. 11 that levels of almost all proinflammatory cytokines were reduced by treatment with in-situ produced engineered EVs showing, once again, that these patient produced EVs are capable of exerting a beneficial therapeutic effect in a disease model.

### Embodiments

1. A composition comprising a delivery vector which comprises a polynucleotide cargo coding for a fusion protein, wherein translation of the polynucleotide cargo into the corresponding fusion protein results in the production of at least one extracellular vesicle (EV) comprising said fusion protein comprising a protein of interest (POI).
2. The composition according to embodiment 1, wherein the delivery vector is a viral vector or a non-viral vector, such as a lipid nanoparticle (LNP), virus like particle (VLP), a cell-penetrating peptide (CPP), a polymer or a pharmaceutically acceptable carrier.
3. The composition according to any one of the preceding embodiments, wherein the polynucleotide cargo is messenger RNA (mRNA), circular mRNA, Doggybone® DNA (dbDNA®), linear DNA, circular DNA, plasmid DNA, linear RNA, circular RNA, self-amplifying RNA or DNA, a viral genome, a modified version of any of the above, or any other polynucleotide cargo.
4. The composition according to any one of the preceding embodiments, wherein the fusion protein comprises at least one EV polypeptide and at least one POI.
5. The composition according to any one of the preceding embodiments, wherein the EV comprising the fusion protein is a patient-derived EV, preferably a liver-cell derived EV, a brain cell-derived EV or a muscle cell-derived EV.
6. The composition according to any one of the preceding embodiments, wherein the fusion protein further comprises at least one targeting domain, at least one endosomal escape domain, at least one cleavable domain, at least one self-cleaving domain, at least one domain binding to a plasma protein, and/or at least one linker.
7. The composition according to any one of the preceding embodiments, wherein the delivery vector is a lipid nanoparticle and the polynucleotide cargo is an mRNA or plasmid DNA.
8. The composition according to any one of embodiments 3-7, wherein the mRNA codes for a fusion protein comprising an EV polypeptide linked to a POI which is an enzyme, optionally wherein the fusion protein further comprises a self-cleaving domain and/or a domain binding to a plasma protein.
9. A pharmaceutical composition comprising the composition according to any one of embodiments 1 to 8 formulated in a pharmaceutically acceptable formulation.
10. The composition according to any one of embodiments 1-8 or the pharmaceutical composition according to embodiment 9 for use in medicine.
11. The composition according to any one of embodiments 1-8 or the pharmaceutical composition according to embodiment 9 for use in the treatment of diseases selected from the group consisting of genetic diseases, lysosomal storage disorders, inborn errors of metabolism, urea cycle disorders, neuromuscular diseases, neurodegenerative diseases, diseases of the central nervous system, kidney diseases, liver diseases, cardiovascular diseases, cancer, infectious disease, autoimmune diseases, and inflammatory diseases.
12. The composition according to any one of embodiments 1-8 or the pharmaceutical composition according to embodiment 9 for use in a method of treatment, the method comprising the step of administering the composition to a target cell of a patient, whereby the target cell of the patient produces patient-derived EVs comprising the fusion protein as a result of translation of the polynucleotide cargo into the corresponding fusion protein.
13. The composition according to any one of embodiments 1-8 or the pharmaceutical composition according to embodiment 9 for use in a method of treatment according to embodiment 12, wherein the target cell is a cell of the liver, the spleen, the lungs, a muscle tissue, the kidneys, the pancreas, the gastrointestinal system, a tissue of the central nervous system including the brain, the bone marrow, a tumour tissue, an immune system cell, and/or any other tissue capable of secreting EVs.
14. A method of producing patient-derived EVs comprising a fusion protein comprising at least one EV polypeptide and at least one POI, the method comprising the step of administering to the cells of a patient the composition according to any one of embodiments 1-8 or the pharmaceutical composition according to embodiment 9, whereby the cells of the patient produces said patient-derived EVs.
15. A patient-derived EV comprising a fusion protein comprising at least one EV polypeptide and at least one POI, wherein said patient-derived EV is manufactured by the method of embodiment 14.
16. The patient-derived EV according to any one of embodiments 14-15, wherein the POI interacts with and transports a protein-based drug cargo and/or RNA-based drug cargo into the EV.
17. The patient-derived EV according to any one of embodiments 14-16 for use in medicine.
18. The patient-derived EV according to any one of embodiments 14-17 for use in the treatment of diseases selected from the group consisting of genetic diseases, lysosomal storage disorders, inborn errors of metabolism, urea cycle disorders, neuromuscular diseases, neurodegenerative diseases, diseases of the central nervous system, kidney diseases, liver diseases, cardiovascular diseases, cancer, infectious disease, autoimmune diseases, and inflammatory diseases.
19. A method of treatment of a disease, disorder or condition in a subject in need thereof, wherein said method comprises administering to a subject a composition according to any one of embodiments 1-8 or the pharmaceutical composition according to embodiment 9, wherein translation of the polynucleotide cargo into the corresponding fusion protein results in the production of at least one EV comprising the fusion protein comprising a POI.
20. A method of treatment of a genetic disease, disorder or condition resulting from a defect gene, said method comprising administering to a subject a composition according to any one of embodiments 1-8 or the pharmaceutical composition according to embodiment 9, wherein translation of the polynucleotide cargo into the corresponding fusion protein results in the production of at least one EV comprising the fusion protein, wherein the POI of the fusion protein is a protein corresponding to the defect gene of the subject.
21. The method according to embodiment 20, wherein the POI is an intracellular or lysosomal enzyme or a membrane protein.
22. The method according to any one of embodiments 20-21, wherein the POI is linked to the EV polypeptide via a self-cleaving polypeptide.
23. The method according to any one of embodiments 20-21, wherein the genetic disease, disorder or condition is an inborn error of metabolism, a urea cycle disorder, a lysosomal storage disorder, a neuromuscular disease, or a neurodegenerative disease.
24. A genetically engineered patient-derived EV, wherein said EV comprises a fusion protein comprising an EV polypeptide and a POI.
25. The genetically engineered patient-derived EV according to embodiment 24, wherein the POI corresponds to a protein that is encoded for by a mutated, deleted, downregulated, or otherwise defect gene of the patient.
26. The genetically engineered patient-derived EV according to any one of embodiments 24-25, wherein the POI is selected from the group consisting of enzymes, transporters, chaperones, transmembrane proteins, structural proteins, nucleic acid-binding proteins, nucleases, recombinases, and protein-binding proteins.
27. The genetically engineered patient-derived EV according to any one of embodiments 24-26, wherein the fusion protein and/or the POI is heterologous to the patient.
28. The genetically engineered patient-derived EV according to any one of embodiments 24-27, wherein the plasma half-life in the patient of a population of the genetically engineered patient-derived EV is more than 2 hours, preferably more than 6 hours, and even more preferably more than 24 hours.
29. The genetically engineered patient-derived EV according to any one of embodiments 24-28, wherein the plasma half-life is measured by assaying plasma for the presence of the fusion protein and/or for the presence of the POI.
30. The genetically engineered patient-derived EV according to any one of embodiments 24-29, wherein the patient-derived EVs are genetically engineered to comprise the fusion protein by translating in a cell of the patient a polynucleotide coding for the fusion protein comprising the EV polypeptide and the POI.
31. The genetically engineered patient-derived EV according to any one of embodiments 24-30, wherein the patient-derived EV is a patient liver cell-derived EV.
32. The genetically engineered patient-derived EV according to any one of embodiments 24-31, for use in medicine.
33. The genetically engineered patient-derived EV according to any one of embodiments 24-32, for use in the treatment of diseases selected from the group consisting of genetic diseases, lysosomal storage disorders, inborn errors of metabolism, urea cycle disorders, neuromuscular diseases, neurodegenerative diseases, diseases of the central nervous system, kidney diseases, liver diseases, cardiovascular diseases, cancer, infectious disease, autoimmune diseases, and inflammatory diseases.

1. A delivery vector comprising a polynucleotide cargo, wherein the polynucleotide cargo codes for a fusion protein comprising a protein of interest (POI) and is arranged to be translated into the fusion protein by an extracellular vesicle (EV)-producing cell, said translation resulting in the production of at least one EV comprising the fusion protein.

2. (canceled)

3. The delivery vector according to claim 1, wherein the polynucleotide cargo is messenger RNA (mRNA), circular mRNA, Doggybone® DNA (dbDNA®), linear DNA, circular DNA, plasmid DNA, linear RNA, circular RNA, self-amplifying RNA or DNA, a viral genome or a modified version of any of the above.

4. The delivery vector according to claim 1, wherein the fusion protein comprises at least one EV polypeptide and at least one POI.

5. The delivery vector according to claim 1, wherein the at least one EV is a patient-derived EV.

6. The delivery vector according to claim 5, wherein the patient-derived EV is a liver cell-derived EV, a brain cell-derived EV or a muscle cell-derived EV.

7. The delivery vector according to claim 1, wherein the fusion protein further comprises at least one targeting domain, at least one endosomal escape domain, at least one cleavable domain, at least one self-cleaving domain, at least one domain capable of binding to a plasma protein and/or at least one linker.

8. The delivery vector according to claim 1, wherein the delivery vector is an LNP and the polynucleotide cargo is an mRNA or plasmid DNA.

9. The delivery vector according to claim 1, wherein the polynucleotide cargo is mRNA, the fusion protein comprises an EV polypeptide linked to the POI and the POI is an enzyme.

10. The delivery vector according to claim 9, wherein the fusion protein further comprises a self-cleaving domain and/or a domain capable of binding to a plasma protein.

11. A pharmaceutical composition comprising a delivery vector according to claim 1.

12. A method of treating a disease or disorder comprising administering to a subject a therapeutically effective amount of a delivery vector according to claim 1.

13. The method according to claim 12, wherein the disease or disorder is a genetic disease, lysosomal storage disorder, inborn error of metabolism, urea cycle disorder, neuromuscular disease, neurodegenerative disease, disease of the central nervous system, kidney disease, liver disease, cardiovascular disease, cancer, infectious disease, autoimmune disease and/or inflammatory disease.

14. A method of treating a disease or disorder in a subject in need thereof, the method comprising administering to the subject a therapeutically effective amount of a delivery vector according to claim 1, a target cell of the patient translating the polynucleotide cargo into the fusion protein, and the target cell producing EVs comprising the fusion protein.

15. The method according to claim 14, wherein the target cell is a liver cell, spleen cell, lung cell, muscle cell, kidney cell, pancreas cell, gastrointestinal cell, central nervous system cell, bone marrow cell, tumour cell, immune system cell or a cell of any other tissue capable of secreting EVs.

16. (canceled)

17. A method of producing EVs in a patient, the method comprising administering a delivery vector according to claim 1 to the patient, and target cells in the patient producing the EVs, wherein the EVs comprise a fusion protein comprising at least one EV polypeptide and at least one POI.

18. (canceled)

19. An EV obtainable by a method according to claim 17.

20. A method of treating a disease or disorder comprising administering to a subject a therapeutically effective mount of an EV according to claim 19.

21. The method according to claim 20, wherein the disease or disorder is a genetic disease, lysosomal storage disorder, inborn error of metabolism, urea cycle disorder, neuromuscular disease, neurodegenerative disease, disease of the central nervous system, kidney disease, liver disease, cardiovascular disease, cancer, infectious disease, autoimmune disease and/or inflammatory disease.

22. A method of treating a disease, disorder or condition in a subject in need thereof, the method comprising administering a delivery vector according to claim 1 to the subject, target cells in the subject translating the polynucleotide cargo into the fusion protein and producing at least one EV, wherein the at least one EV comprises a fusion protein comprising a POI.

23. The method according to claim 22, wherein the disease, disorder or condition is a genetic disease, disorder or condition resulting from a defective gene and the POI is a protein corresponding to the defective gene.

24. The method according to claim 23, wherein the POI is an intracellular or lysosomal enzyme or a membrane protein.

25. The method according to claim 23, wherein the fusion protein comprises the POI linked to an EV polypeptide via a self-cleaving polypeptide.

26. (canceled)

27. A genetically engineered patient-derived EV, wherein the EV comprises a fusion protein comprising an EV polypeptide and a POI.

28. (canceled)

29. The genetically engineered patient-derived EV according to claim 27, wherein the POI is an enzyme, transporter, chaperone, transmembrane protein, structural protein, nucleic acid-binding protein, nuclease, recombinase and/or protein-binding protein.

30. The genetically engineered patient-derived EV according to claim 27, wherein the fusion protein and/or the POI is heterologous to the patient.

31. The genetically engineered patient-derived EV according to claim 27, wherein the EV has a plasma half-life in the patient of more than two hours, more than six hours or more than 24 hours.

32-34. (canceled)

35. A method of treating a disease or disorder comprising administering to a subject a therapeutically effective amount of a genetically engineered patient-derived EV according to claim 27.

36. (canceled)
